SEQUENCE LISTING (6 sequences)

SEQ ID NO: 1 - DNA, 23 bp, synthetic construct
  source 1..23: mol_type = other DNA
ggtaagagatcaggctgaacatc

SEQ ID NO: 2 - DNA, 20 bp, synthetic construct
  source 1..20: mol_type = other DNA
agacagcagtacaaatggca

SEQ ID NO: 3 - DNA, 20 bp, synthetic construct
  source 1..20: mol_type = other DNA
ccccaatccccccttttctt

SEQ ID NO: 4 - DNA, 22 bp, synthetic construct
  source 1..22: mol_type = other DNA
ctgctgtccctgtaataaaccc

SEQ ID NO: 5 - DNA, 24 bp, synthetic construct
  source 1..24: mol_type = other DNA
agtgcaggggaaagaatagtagac

SEQ ID NO: 6 - DNA, 300 bp, synthetic construct
  source 1..300: mol_type = other DNA
tagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaa

(19) United States
(12) Patent Application Publication
(10) Pub. No.: US 2026/0219263 A1
(43) Pub. Date: Jul. 30, 2026

(54) CAMOUFLAGING ON CHIP, METHODS, AND SYSTEMS AND ITS USE FOR TESTING BIOLOGICAL AND NON-BIOLOGICAL ANALYTES

(71) Applicant: CASE WESTERN RESERVE UNIVERSITY, Cleveland, OH (US)

(72) Inventor: Mohamed S. Draz, Cleveland, OH (US)

(21) Appl. No.: 19/146,006

(22) PCT Filed: Jan. 5, 2024
(86) PCT No.: PCT/US2024/010511

§ 371 Date: Jul. 7, 2025

Related U.S. Application Data
(60) Provisional application No. 63/478,623, filed on Jan. 5, 2023.

(51) Int. Cl. G01N 33/543; G01N 21/78; G01N 33/58

(52) U.S. Cl. CPC G01N 33/54313; G01N 21/78; G01N 33/587

(57) ABSTRACT

An apparatus for detection of an analyte includes a first detectable agent having one or more detectable characteristic and masking agent having one or more characteristic that masks the detection of the one or more characteristic of the first detectable agent in absence of the analyte, wherein at least one of the first detectable agent or the masking agent is configurable in the presence of the analyte to allow detection of the one or more characteristic of the first detectable agent which is indicative of the presence of the analyte.

### RELATED APPLICATION

This application claims priority from U.S. Provisional Application No. 63/478,623, filed Jan. 5, 2023, the subject matter of which is incorporated herein by reference in its entirety.

### GOVERNMENT FUNDING

This invention was made with government support under CS254566, DA054557, and TWO12056 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Jan. 5, 2024, is named CWR-032063WO ORD st.26 and is 6,568 bytes in size.

### BACKGROUND

Current devices and system for multiplex analyte testing typically require staining or multistep labeling that is necessarily performed using remote laboratory testing. Additionally, in current analyte sensing approaches that utilize color (e.g., ELISA) and fluorescence (e.g., PCR) labels, the generated color or fluorescence detection signal is generally diffused over a relatively large volume of sample that leads to lower detection sensitivity and sensing efficiency. Also, these current approaches may not enable efficient integration with AI-based image analysis systems or even simple optical systems with a camera. There remains a need for novel devices allowing for simpler methods and systems for rapid detection with high multiplexity, e.g., in point-of-care (POC) diagnostics, that can detect the target analyte with different modalities, such as color, fluorescence, etc., without the need for staining or multistep labeling.

### SUMMARY

This disclosure describes an apparatus for analyte detection as well as methods and systems using the apparatus in the detection of an analyte. Advantageously, the apparatus allows for bioinspired camouflaging for sensing and testing biological and non-biological analytes in samples with high multiplexity at the level of the target by detecting different targets, and at the level of modality by detecting different modalities, such as color and/or fluorescence.

The apparatus for detection of an analyte includes a first detectable agent having one or more detectable characteristic. The apparatus also includes a masking agent having one or more characteristic that masks the detection of the one or more characteristic of the first detectable agent in absence of the analyte. At least one of the one or more characteristic of the first detectable agent or the masking agent is configurable in the presence of the analyte to allow detection of the one or more characteristic of the first detectable agent which is indicative of the presence of the analyte.

In some embodiments, the first detectable agent is configured to specifically bind to the analyte. The one or more characteristics of the first detectable agent can be undetectable when the first detectable agent is not bound to the analyte and detectable upon binding to the analyte.

In some embodiments, binding of the analyte to the first detectable agent can trigger configuration of the masking agent to form an unmasked or mask-free signal zone that allows detection of the first detectable agent.

In some embodiments, the masking agent may include a masking fluid that masks the one or more characteristic of the first detectable agent. In some embodiments, binding of the analyte to the first detectable agent forms a mask-free zone indicative of the presence of the analyte. In some embodiments, binding of the analyte to the first detectable agent disperses masking fluid around the first detectable agent to allow detection of the first detectable agent, which is indicative of the presence of the analyte.

In some embodiments, the first detectable agent can include one or more first detectable particles affixed to a surface of a substrate. The first detectable particles can be configured to specifically bind to analyte. In some embodiments, the one or more first detectable particles affixed to the surface of the substrate are covered with the masking fluid in an absence of the analyte.

In some embodiments, the apparatus further includes a detection agent that specifically binds to the analyte to from a detection agent-analyte-particle complex. The detection agent can be masked when the analyte is not bound to the detectable particle and the detection agent.

In some embodiments, the masking fluid visually masks the first detectable particle(s) that do not form the detection agent-analyte-particle complex. The detection agent of the complex can catalyze conversion of the masking fluid into a gas that disperses masking fluid around the complex to form the fluid-free zone and allowing optical detection of the complex on the substrate.

In some embodiments, the first detectable particle(s) include a plurality of microbeads and can have a diameter of about 10 μm to about 1 mm.

In some embodiments, the first detectable particles include a capturing agent on outer surfaces of the first detectable particles that specifically bind to the analyte. The capturing agent can include at least one of a small molecule, nucleotide, protein, antibody, or streptavidin. In some embodiments, the capturing agent is conjugated to the first detectable particle outer surface using a linker.

In some embodiments, the capturing agent can include streptavidin and the analyte includes a biotin-modified nucleic acid or peptide. The biotin-modified nucleic acid analyte can include a multi-arm amplicon nucleic acid structure. The multi-arm amplicon structure is formed using a multi-arm primer, the multi-arm primer includes sense and anti-sense arms of single stranded oligonucleotides that are linked to a core by a flexible linker. The anti-sense arm(s) complement(s) an anti-sense sequence of a target nucleic acid and the sense arm(s) complement(s) a sense sequence of the target nucleic acid distinct from and subsequent to the anti-sense sequence.

In some embodiments, the detection agent can include platinum nanoparticles (PtNPs). The PtNPs can be conjugated to streptavidin, or anti-biotin antibodies.

In some embodiments, the masking fluid can have a color that visually masks the first detectable particles(s) appearance upon loading of the masking fluid onto the surface of the substrate. In some embodiments, the masking fluid can include a peroxide solution, for example a peroxide solution having about 1% to about 20% hydrogen peroxide (w/v), about 5% to about 15% hydrogen peroxide (w/v), or about 10% hydrogen peroxide (w/v).

In some embodiments, the substrate can include at least one microchannel that extends along a portion of the substrate. The microchannel can include the first detectable particle(s) and masking fluid. The microchannel may include a fluid inlet and an outlet for receiving fluids and removing fluid from the microchannel.

In some embodiments, the apparatus can further include one or more second detectable agents. The second detectable agent can include second detectable particles affixed to the surface of the substrate. The second detectable particles can include a second plurality of second capture agents to specifically bind a second analyte. The apparatus can further include a plurality of second detection agents that include a second targeting moiety to specifically bind the second analyte. Binding of the second analyte to both the second detectable particles and the second detection agent may form a second detection agent-analyte-particle complex that forms a fluid-free zone around the second agent-analyte-particle complex allowing for detection of the second agent-analyte-particle complex on the substrate.

In some embodiments, in order to optically differentiate between detection of a first analyte and a second analyte in a sample using the apparatus, the one or more second detectable particles(s) are a substantially different color than the first detectable particle(s). For example, the one or more first detectable particles can be red and the one or more second detectable particles can be blue to easily determine the presence of one or both analytes in a fluid sample.

In some embodiments, the first analyte and/or second analyte, or first analyte of interest and/or second analyte of interest, is a component of a biological fluid sample. The biological fluid sample may be obtained from a subject, such as a human subject. In particular embodiments, the apparatus is a point-of-care (POC) device.

Other embodiments described herein relate to a method of detecting analyte. The method includes providing an apparatus described herein. A sample including the analyte is loaded onto the surface of the substrate to bind the analyte to the outer surface of one or more first detectable particles. The bound analyte is labeled with the plurality of first detection agents to form a first detection agent-analyte-particle complex on the surface of the substrate. A masking fluid is loaded onto the surface of the substrate to mask the one or more first detectable particle(s) of the complex. The detection agent of the complex can then catalyze degradation of the masking fluid to form a masking fluid-free zone around the particle of the complex. The one or more first detectable particle(s) of the complex in the masking fluid-free zone can then be detected. Detection of first detectable particle(s) of the complex in the masking fluid-free zone is indicative of the presence of the analyte in the sample.

In some embodiments, the method can further include a washing step to remove uncaptured analyte and the remaining sample from the surface of the substrate prior to labeling the bound analyte with the plurality of first detection agents.

In some embodiments, the step of optically detecting the one or more particle(s) can include imaging the surface of the substrate with a camera.

In some embodiments, the camera that is part of a mobile device, such as a cellular phone.

In some embodiments, the method may further include determining if there is an optically first detectable particle in at least one image in a masking fluid-free zone. The number of particle(s) and/or the size of a masking fluid-free zone(s) optically detected is indicative of the concentration of analyte in the sample.

In some embodiments, the sample can include two or more different analytes and the apparatus is configured to detect two or more different analytes from the sample. In certain embodiments, the method can further include the step of contacting a nucleic acid analyte in the fluid sample with a set of biotin-modified nucleic acid primers specific for the nucleic acid analyte to form a biotinylated nucleic acid target analyte, wherein the biotinylated nucleic acid target analyte is bound to the outer surface of the one or more first detectable particle(s) by streptavidin conjugated to the particle(s).

In some embodiments, the biotin-modified nucleic acid analyte can include a multi-arm amplicon nucleic acid structure and the multi-arm amplicon structure can be formed using a multi-arm primer. The multi-arm primer can include sense and anti-sense arms of single stranded oligonucleotides that are linked to a core by a flexible linker, wherein the anti-sense arm(s) complement(s) an anti-sense sequence of a target nucleic acid and the sense arm(s) complement(s) a sense sequence of the target nucleic acid distinct from and subsequent to the anti-sense sequence. The sense arm(s) are linked to the anti-sense arm(s) with a multi-arm polyethylene glycol (PEG) linker, and each arm of the multi-arm PEG linker is connected to either the sense arm of a single strand oligonucleotide or the anti-sense arm of a single oligonucleotide.

In some embodiments, the method further includes the step of amplifying a target nucleic acid analyte by providing a multi-arm primer and annealing the multi-arm primer to the target nucleic acid analyte.

In some embodiments, after annealing to the nucleic acid target sequence, the primer can be extended using a polymerase having high strand displacement activity. The polymerase having high strand displacement activity can be selected from the group consisting of Bst DNA polymerase and Phi29 (<29) DNA polymerase.

In some embodiments, the multi-arm primer includes at least two sense arms and at least two anti-sense arms. The anti-sense arms can be configured to bind to the complementary sequence in the nucleic acid and form looped-arm dsDNA structure mediated by the sense arms. The anti-sense arms can prime for amplification, followed by self-priming with sense arms generating a stem-looped DNA hybrid structure in which loops are partially made of the sense arm and anti-sense arm and the linker. Other free anti-sense arms and sense arms of the multi-arn primer can mediate a chain of self-looping to form multi-arm amplicons, which are used as templates to generate large multi-amplicon structures and self-prime for the formation of new strand and stem-looped structures. The self-looping can continue until all the sense arms expand into a copy of the target sequence.

Other embodiments described herein relate to a system that includes the apparatus described herein for performing the method of detecting the analyte. The system further includes a camera, a processor, and one or more non-transitory computer readable media to store executable instruction and data. The processor, such as a microprocessor, can access the media and execute the instructions to perform the method of detecting the analyte. The executable instructions can include a camera interface configured to instruct the camera to capture at least one image. In exemplary embodiments, the camera can be part of a mobile device, such as a cellular phone.

Other objects and advantages and a fuller understanding of the invention will be had from the following detailed description and the accompanying drawings.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIGS. 1(A-B) illustrate CamoChip concept and design in accordance with an embodiment described herein. (A) is an image showing a camouflaging of a blue microbead in a drop of a blue peroxide solution on a glass slide. The inset image shows the drop prior to the formation of a fluid-free (clear) zone around the bead making it visible to the naked eye. (B) is a schematic presentation of the design of CamoChip using HIV-1 RNA. The bead is modified with streptavidin to capture biotinylated viral RNA that induces the accumulation of streptavidin (SA)-platinum nanoparticles (PtNPs), which in turn catalyzes the degradation of the surrounding peroxide blue solution, creating a clear area around the blue bead.

FIGS. 2 (A-B) illustrate CamoChip technology on a slide in accordance with an embodiment described herein. (A) is a digital image of decamouflaged bead on a glass slide, and the formed clear area around the bead. (B) Bead decamouflaging at different time points. The formation of the clear area around the bead starts as early as 2 seconds and continue to increase in size until the formation of a circular clear zone of 1.5-3 mm, making the masked bead visible to the naked eye.

FIG. 3 illustrates a digital image of a CamoChip microchip apparatus in accordance with an embodiment described herein for HIV-1 RNA analyte detection.

FIGS. 4(A-B) illustrate additional digital images of a CamoChip microchip apparatus in accordance with an embodiment described herein for HIV-1 RNA analyte detection. The biotinylated HIV-1 RNA is loaded on a chip with streptavidin-modified blue beads. The chip is incubated for 10 minutes to capture the target RNA then followed by streptavidin-platinum nanoparticles to label the captured RNA. A blue in color peroxide solution is loaded on the chip to mask the beads. After 5 minutes incubation, the accumulated PtNPs on beads degrade the surrounding peroxide solution, creating a clear area around the beads. (A) The microchip loaded with the peroxide solution at 0 minutes time. (B) The microchip loaded with the peroxide solution after 5 minutes incubation at room temperature.

FIG. 5 illustrates a digital image of a CamoChip microchip apparatus in accordance with an embodiment described herein for HIV-1 RNA analyte detection at different time points from 0 minutes time to 15 minutes.

FIG. 6 is a digital image of a CamoChip microchip apparatus in accordance with an embodiment described herein for multiplex detection of HIV-1 RNA on blue colored beads and HIV-1 core antigen (on red colored beads).

FIGS. 7(A-B) are digital images showing CamoChip technology results for samples with HIV-1 RNA only (no HIV-1 core antigen). (A) Digital image of the microchip after the target RNA capture and labeling with PtNPs and before loading the camouflaging peroxide solution. (B) The formation of clear areas on microchip after loading the camouflaging peroxide solution and incubation for 5 minutes.

FIGS. 8(A-B) are digital images showing CamoChip technology results for samples with HIV-1 core antigen only (no HIV-1 core antigen). (A) Digital image of the microchip after capture of HIV-1 core Ag and labeling with PtNPs and before loading the camouflaging peroxide solution. (B) The formation of clear areas on microchip after loading the camouflaging peroxide solution and incubation for 5 minutes.

FIG. 9 illustrates a digital image of a CamoChip microchip apparatus in accordance with an embodiment described herein for multiplex testing of samples with HIV-1 core antigen and HIV-1 RNA at different time points from before sample loading to 10 minutes after sample loading and signal formation.

FIG. 10 illustrates a digital image showing the detection sensitivity of a CamoChip microchip apparatus in accordance with an embodiment described herein using HIV-1 RNA samples.

FIG. 11 illustrates digital images of co-infection detection of HIV-1/HBV using CamoChip microchip apparatus in accordance with an embodiment described herein. HBV particles are captured and detected on red colored beads and HIV-1 RNA is captured and detected on blue colored beads.

FIG. 12 is an illustration and digital image of the use of CamoChip microchip apparatus in combination with cellphone technology in accordance with an embodiment described herein. A cellphone enabled with AI-based algorithm trained for detecting the color, size and the diameter of clear zone formations can be used for quantitative testing of multiple targets at the point-of-care (POC).

FIG. 13 is a schematic illustration in accordance with an embodiment described herein showing a pattern made by the first agent, e.g., polymers, magnetic material, crystals, that cover the first agent, or first object. In response to an analyte of interest, the patterned, patterned-like, or non-patterned presence of the second agent is changed, or altered, in a manner allowing for the first agent to become detectable, e.g., through visual or non-visual detection.

FIG. 14 is a schematic illustration of the detection of target analyte using an apparatus in accordance with an embodiment described herein. An emulsion of two or three phases is loaded onto a substrate surface including a number of optically visible beads such that the emulsion creates a pattern that mask, or camouflage, the beads. Upon introduction of a sample including a target analyte, the target induces a reaction that converts the emulsion into a non-emulsion form and separate. Separation of the emulsion unmask, or decamouflage, the beads, thereby allowing for visible detection of the beads.

FIGS. 15 (A-B) are digital images showing an apparatus in accordance with an embodiment described herein. (A) Different colors codes are laser printed on the surface of plastic sheets. Each color codes for a specific target analyte and configured to hold a specific volume of sample to maximize testing sensitivity and overall performance of the apparatus. (B) Fully assembled CamoChip apparatus, fully flexible, lightweight, and low cost.

FIG. 16 is a flow diagram illustrating a method of detecting an analyte.

FIGS. 17(A-B) illustrate CamoChip design and fabrication. (A) Microchips are fabricated using polymethyl methacrylate (PMMA), double-sided adhesive (DSA), and a glass slide. First, colored beads are partially embedded on the glass surface of microchip using a layer of liquid plastic. Then the PMMA layer with the inlet and outlet assembled on the glass slide using DSA layer that forms a single microfluidic channel with the colored beads. (B) Digital images show the actual CamoChip with beads before (i) and after (ii) adding the camouflaging fluid with blue color.

FIGS. 18(A-E) illustrate beads surface functionalization and characterization. (A) Bright-field image shows solid spherical beads with two colors, blue and red and average size of 289.1±42.33 μm. (B) Fluorescence spectroscopy analysis of anti-hapten modified beads. Anti-hapten functionalized beads are stained with FITC-G protein that specifically interact with IgG on the surface of beads. The inserted bar chart shows the concentration of IgG protein on the surface of functionalized beads, measured using UV-vis spectroscopy. (C) SDS-PAGE analysis of anti-hapten mAb released from the surface of beads. Lane M: protein marker; Lane 1: beads—functionalized with mAb; Lane 2: mAb. (D) FT-IR spectra of surface functionalized beads with anti-hapten mAb. (E) SDS-PAGE analysis of streptavidin (SA)-functionalized beads. Lane M: protein marker; Lane 1: beads-functionalized with SA; Lane 2: SA.

FIGS. 19(A-D) illustrate Pt-nanoprobes preparation and characterization. (A) Schematic of Pt-nanoprobes preparation. Anti-hapten IgG monoclonal antibody was coupled to PtNPs through a hydrazide reactive crosslinker of PDPH (3-(2-pyridyldithio)propionyl hydrazide) that has a terminal pyridinethiol. The reduced PDPH has a free terminal thiol group that binds to the surface of the PtNPs, forming hydrazide functionalized PtNPs that can react with the carbohydrate residue of the oxidized antibody. (B) Characterization of PtNPs: TEM image and particle size distribution histogram. (C) UV-vis absorption spectra of PtNPs (citrate capped platinum nanoparticles, black) and Pt-nanoprobe with mAb (platinum nanoparticles conjugated to IgG, blue). (D) Agarose gel electrophoresis of PtNPs (unmodified platinum nanoparticles) and Pt-nanoprobes with IgG (Anti-hapten antibody-conjugated platinum nanoparticles, red).

FIGS. 20(A-C) illustrate CamoChip design testing and optimization. (A) CamoChip with blue beads masked (camouflaged) and unmasked (decamouflaged) after incubation for 600 s with blue camouflage fluid of 10% peroxide. (B) CamoChip at different time points (0-300 s) of incubation. (C) The increase in the efficiency of decamouflaging beads (i) and diameter of the formed clear zone (ii) at different time points.

FIGS. 21(A-B) illustrate the performance of CamoChip in HIV-1 RNA testing in PBS-spiked samples. (A) Specificity testing of CamoChip using the targeted HIV-1 RNA and non-targeted viral nucleic acid of HBV and HCV at high concentration of 10⁶ copies/ml. Representative images of microchips associated with HIV and the tested non-specific targets of HBV and HCV. (B) Sensitivity testing using serial dilutions of HIV-1 RNA spiked in PBS buffer at concentrations that range from 10⁰ to 10⁹ copies/ml. The target HIV-1 RNA is incubated with biotinylated DNA probes, captured and labeled with Pt-nanoprobes on the surface of beads on chip. The decamouflage efficiency is calculated from the number of unmasked beads from the total number of beads included on the surface of each chip, after incubation time of 5 minutes. Representative images of microchips associated with HIV RNA-spiked PBS samples testing.

FIG. 22 illustrates the performance of CamoChip in HIV-1 RNA testing in PBS and Plasma-spiked samples.

FIGS. 23(A-B) illustrate the performance of CamoChip in multiplex testing of HIV-1 RNA and HIV-1 p24 in PBS-spiked samples. (A) The decamouflage efficiency of beads (bars) and the diameter of clear zones (circle) formed in the presence of HIV-1 RNA mixed with and without HIV-1 p24 antigen. (B) Representative images of microchip associated with HIV-spiked PBS samples of different RNA concentrations. Blue beads encode for HIV-1 RNA and red ones encode for p24.

FIG. 24 illustrates target sequence (SEQ ID NO: 6) and the design of the MAPA primers. Initiator primer (IP) that is 23-mer in length with a Tm value of 55.3° C. Looping primer (LP) comprises two sequences; each is 20-mer in length: looping sequence 1 (L1) with a Tm value of 54.5° C. and looping sequence 2 (L2) with a Tm value of 56.5° C. Unlooping primer (ULP) that is 22-mer in length with a Tm value of 55.5° C. Arrowheads indicate the direction of DNA synthesis, and the dashed line shows the looping site and step.

FIGS. 25(A-C) illustrate the multi-arm primed amplification (MAPA) reaction of nucleic acid. (A) starting step of MAPA reaction and the formation of the looped-arm amplicons that serve as a template for the arm extension through the cycling step and the formation of multi-arm amplicons. (B) cycling step that involves the expansion of each PEG-DNA arm into a copy of the target sequence through a cycle of arm looping and unlooping steps. (C) Elongation step and the formation of large multi-amplicon structures.

FIGS. 26(A-D) illustrate multi-arm primer synthesis and characterization. (A) synthesis of the multi-arm polyethylene glycol (PEG)-DNA primer. Branched PEG molecules with eight side chains (each as an arm) in total and six maleimide-modified chains are coupled with a 1:1 mixture of thiolated looping sequences 1 and 2 (L1 and L2) to form a multi-arm looping primer (LP). (B, C) Characterization of the synthesized multi-arm DNA primers using magnetic bead separation coupled with agarose gel electrophoresis. (i) Schematic presentation for the used protocol, in which streptavidin beads are incubated with the synthesized biotinylated muti-arm primers to form MB-SA/biotin-PEG-DNA complex that is subsequently loaded into an agarose gel. (ii) Electrophoretic analysis of the formed MB-PEG/DNA complex using 0.5% agarose gel electrophoresis. (D) UV-vis spectroscopy analysis of the formed multi-arm DNA primers.

FIG. 27 illustrates testing the primer design and the working protocol of MAPA. The amplification products of multiple working protocols with the designed muti-arm looping primer (LP), its composing sequences of looping sequence 1 (L1) and looping sequence 2 (L2), and other primers, including initiator primer (IP), unlooping primers (ULP and ULP′), were tested. The amplification reaction was performed at 65 C for 20-35 minutes, and the formed amplification products were tested using 2% agarose gel.

FIG. 28 illustrates optimization of the MAPA method. The formation of specific DNA amplicons (in the presence of the target nucleic acid) using MAPA was tested at reaction temperatures ranging from 65° C. to 69° C. and reaction times up to 35 minutes. NC: negative control (no target nucleic acid is added), M: marker DNA.

FIG. 29 illustrates detection sensitivity of the developed MAPA method. Electrophoresis patterns of MAPA amplification products generated from different concentrations of HIV-1 target DNA plasmid. MAPA reaction was performed using 10-fold serial dilutions of the HIV-1 DNA template (1010 copies/mL-100 copies/mL) at 67° C. for 30 minutes. M: 1-kb DNA ladder marker; NC: negative control (without target DNA template).

FIGS. 30(A-B) illustrate potential applications of MAPA for target amplification and testing. (A) MAPA amplification on magnetic beads (MBs) compared with control (MAPA without magnetic beads). The optimized MAPA protocol was performed for HIV-1 testing in the presence of streptavidin-MBs, and the formed biotinylated MAPA amplicons (due to the use of biotinylated PEG in the synthesis of MAPA LP) are spontaneously captured on beads and can be readily separated on an agarose gel or by an external magnetic field. (B) Testing of MAPA amplicons after magnetic separation using gel electrophoresis and fluorescence spectroscopy.

FIG. 31 is a schematic diagram illustrating a system using an apparatus in accordance with an embodiment described herein.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also includes the general class of which a specific example may be used for illustration. The terminology herein is used to describe specific aspects of the invention, but their usage does not delimit the invention, except as outlined in the claims.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation. “Optional” or “optionally” means that the subsequently described circumstance may or may not occur, so that the description includes instances where the circumstance occurs and instances where it does not.

The term “microchannels” as used herein refer to pathways through a medium, e.g., plastic, silicon or glass, that allow for movement of liquids and gasses. Microchannels can therefore connect other components, i.e., keep components “in fluid communication.” While it is not intended that the present application be limited by precise dimensions of the channels, illustrative ranges for channels are as follows: the channels can be between 0.1 and 100 μm in depth (e.g., 50 μm) and between 50 and 10,000 μm in width (e.g., 400 μm). The channel length can be between 1 mm and 100 mm (e.g., about 27 mm).

The term “polymer” as used herein refers to a substance formed from two or more molecules of the same substance. Polymers may also be linear polymers in which the molecules align predominately in chains parallel or nearly parallel to each other. In a non-linear polymer, the parallel alignment of molecules is not required.

The term “lens less image” or “lens less mobile imaging system” as used herein refers to an optical configuration that collects an image based upon electronic signals as opposed to light waves. For example, a lens less image may be formed by excitation of a charged coupled device (CCD) sensor by emissions from a light emitting diode.

The term “charge-coupled device (CCD)” as used herein refers to a device for the movement of electrical charge, usually from within the device to an area where the charge can be manipulated, for example, a conversion into a digital value. A CCD provides digital imaging when using a CCD image sensor where pixels are represented by p-doped MOS capacitors.

The term “patient” or “subject” as used herein is a human or animal and need not be hospitalized. For example, out-patients, persons in nursing homes are “patients.” A patient may comprise any age of a human or non-human animal and therefore includes both adult and juveniles, i.e., children. It is not intended that the term “patient” or “subject” connote a need for medical treatment and, thus, a patient, or subject, may voluntarily or involuntarily be part of experimentation whether clinical or in support of basic science studies.

The term “functionalized” or “chemically functionalized” as used herein means the addition of functional groups onto the surface of a material by chemical reaction(s). As will be readily appreciated by a person skilled in the art, functionalization can be employed for surface modification of materials in order to achieve desired surface properties, such as biocompatibility, wettability, and so on. Similarly, the term “biofunctionalization,” “biofunctionalized,” or the like, as used herein, means modification of the surface of a material to have desired biological function, which will he readily appreciated by a person of skill in the related art, such as bioengineering.

The term “sample” as used herein is used in its broadest sense and includes environmental and biological samples. Environmental samples include material from the environment such as soil and water. Biological samples may be animal, including, human, fluid, e.g., blood, plasma, and serum; solid, e.g., stool; tissue; liquid foods, e.g., milk; and solid foods, e.g., vegetables. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The terms “capturing agent”, “bioaffinity ligand”, “binding component”, “ligand” or “receptor” as used herein may be any of a large number of different molecules, biological cells or aggregates, and the terms are used interchangeably. Each capturing agent may be immobilized on a solid substrate and binds to an analyte being detected. Proteins, polypeptides, peptides, nucleic acids (nucleotides, oligonucleotides and polynucleotides), antibodies, ligands, saccharides, polysaccharides, microorganisms such as bacteria, fungi, and viruses, receptors, antibiotics, test compounds (particularly those produced by combinatorial chemistry), plant and animal cells organdies or fractions of each and other biological entities may each be a capturing agent. Each, in turn, also may be considered as analytes if same bind to a capturing agent.

The terms “bind” or “adhere” as used herein include any physical attachment or close association, which may be permanent or temporary. Generally, an interaction of hydrogen bonding, hydrophobic forces, van der Waals forces, covalent and ionic bonding etc., facilitates physical attachment between the capturing agent and the analyte being measured. The “binding” interaction may be brief as in the situation where binding causes a chemical reaction to occur. That is typical when the binding component is an enzyme, and the analyte is a substrate for the enzyme. Reactions resulting from contact between the capturing agent and the analyte are also within the definition of binding for the purposes of this application.

The terms “DNA template”, or “template” as used herein, refer to a nucleic acid that is used by a polymerase to synthesize a new complementary nucleic acid.

The term “oligonucleotide” as used herein is defined as a molecule comprised of two or more deoxyribonucleotides or ribonucleotides. The exact size will depend on many factors, which in turn depends on the ultimate function or use of the oligonucleotide. Oligonucleotides can be prepared by any suitable method, including, for example, cloning and restriction of appropriate sequences and direct chemical synthesis by a method such as the phosphotriester method, the diethylphosphoramidite method, and the solid support method. A review of synthesis methods is provided in [Goodchild J., Bioconjug. Chem. V.l (1990), P. 165-187].

The term “primer”, as used herein, means an oligonucleotide, either natural or synthetic that is capable, upon forming a duplex with a polynucleotide template, of acting as a point of initiation of nucleic acid synthesis when placed under conditions in which primer extension is initiated and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. In some embodiments, synthesis of a primer extension product, which is complementary to a nucleic acid strand, is initiated in the presence of the requisite four different nucleoside triphosphates and a thermostable DNA polymerase in an appropriate buffer at a suitable temperature. A “buffer” includes cofactors (such as divalent metal ions) and salt (to provide the appropriate ionic strength), adjusted to the desired pH.

Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Strand displacement activity”, as used herein, refers to the phenomenon by which an enzyme, such as a DNA polymerase, causes the dissociation of a paired nucleic acid from its complementary strand in a direction from 5′ towards 3′, in conjunction with, and close to, the template-dependent nucleic acid synthesis. The strand displacement starts at the 5′ end of a paired nucleic acid sequence and the enzyme therefore carries out the nucleic acid synthesis. The neosynthesized nucleic acid and the displaced nucleic acid generally have the same nucleotide sequence, which is complementary to the template nucleic acid strand. The strand displacement activity may be situated on the same molecule as that conferring the activity of nucleic acid synthesis, and particularly the DNA synthesis. “Strong strand displacement activity” of DNA polymerase, as used herein, allows use of the enzyme for carrying out isothermal polymerase reactions like that of Bst polymerase.

“Amplicon” means the product of a polynucleotide amplification reaction; that is, a clonal population of polynucleotides, which may be single stranded or double stranded, which are replicated from one or more starting sequences. “Amplifying” means producing an amplicon by carrying out an amplification reaction. The one or more starting sequences may be one or more copies of the same sequence, or they may be a mixture of different sequences. Preferably, amplicons are formed by the amplification of a single starting sequence. Amplicons may be produced by a variety of amplification reactions whose products comprise replicates of the one or more starting, or target, nucleic acids. In some embodiments, amplification reactions producing amplicons are “template-driven” in that base pairing of reactants, either nucleotides or oligonucleotides, have complements in a template polynucleotide that are required for the creation of reaction products. In some embodiments, template-driven reactions are primer extensions with a nucleic acid polymerase or oligonucleotide ligations with a nucleic acid ligase.

As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Bioassay reagents” or “assay reagents,” which are used interchangeably herein, mean reagents used to perform an analytical reaction on a cartridge of the invention. Such reagents may include enzymes, enzyme co-factors, primers, waxes for bubble suppression, salts, buffers, solvents, agents to modify the secondary structure of analytes, labels (such as fluorescent dyes or fluorescently labeled oligonucleotides), lysis buffers, and the like, which make up a reaction mixture. In some embodiments, assay reagents include reagents for carrying out an isothermal amplification of one or more target polynucleotides.

“Isothermal amplification” in reference to an assay to amplify a target nucleic acid or polynucleotide means a method of replicating a target nucleic acid without a requirement of thermal cycling. That is, without a requirement of subjecting a reaction mixture to cycles of different temperatures in order to melt target nucleic acid strands, anneal primers and provide for extension conditions for a DNA polymerase. An isothermal amplification is typically performed at a predetermined temperature.

A “microfluidic”, “microfluidics” device or “nanofluidics” device, used interchangeably herein, each means an integrated system for capturing, moving, mixing, dispensing or analyzing small volumes of fluid, including samples (which, in turn, may contain or comprise cellular or molecular analytes of interest), reagents, dilutants, buffers, or the like. Generally, reference to “microfluidics” and “nanofluidics” denotes different scales in the size of devices and volumes of fluids handled. In some embodiments, features of a microfluidic device have cross-sectional dimensions of less than a few hundred square micrometers and have passages, or channels, with capillary dimensions, e.g., having maximal cross-sectional dimensions of from about 1-2 mm to about 0.1 pm. In some embodiments, microfluidics devices have volume capacities in the range of from 100 pL to a few nL, e.g., 10-100 nL or in the range of from 100 pL to 1 pL. Dimensions of corresponding features, or structures, in nanofluidics devices are typically from 1 to 3 orders of magnitude less than those for microfluidics devices. One skilled in the art would know from the circumstances of a particular application which dimensionality would be pertinent. In some embodiments, microfluidic or nanofluidic devices have one or more chambers, ports, and channels that are interconnected and in fluid communication and that are designed for carrying out one or more analytical reactions or processes, either alone or in cooperation with an appliance or instrument that provides support functions, such as sample introduction, fluid and/or reagent driving means, such as positive or negative pressure, acoustical energy, or the like, temperature control, detection systems, data collection and/or integration systems, and the like. In some embodiments, microfluidics and nanofluidics devices may further include valves, pumps, filters and specialized functional coatings on interior walls, e.g., to prevent adsorption of sample components or reactants, facilitate reagent movement by electroosmosis, or the like. Such devices may be fabricated as an integrated device in a solid substrate, which may be glass, plastic, or other solid polymeric materials, and may have a planar format for ease of detecting and monitoring sample and reagent movement, especially via optical or electrochemical methods. In some embodiments, such devices are disposable after a single use.

This disclosure describes an apparatus for analyte detection as well as methods and systems using the apparatus in the detection of an analyte. Advantageously, the apparatus allows for bioinspired camouflaging for sensing and testing biological and non-biological analytes in samples with high multiplexity at the level of the target by detecting different targets, and at the level of modality by detecting different modalities, such as color and/or fluorescence.

In some embodiments, the apparatus for detection of an analyte includes a first detectable agent having one or more detectable characteristic. The apparatus also includes a masking agent having one or more characteristic that masks, e.g., camouflages, the detection of the one or more characteristic of the first detectable agent in the absence of an analyte of interest. In the presence of an analyte of interest, the first detectable agent or the masking agent is configurable to allow detection of the one or more characteristic properties of the first detectable agent, e.g., decamouflage, thereby indicating the presence of the analyte.

In some embodiments, the first detectable agent and/or masking agent may bind to an analyte when placed in the presence of the analyte. For example, the first detectable agent can be configured to specifically bind to an analyte of interest. The one or more characteristic of the first detectable agent can be undetectable in the presence of the masking agent when the first detectable agent is not bound to the analyte and detectable when bound to the analyte.

Detection of an analyte using the apparatus described herein can be determined by the formation of an unmasked or mask-free-signal zone that allows detection of the first detectable agent. In certain embodiments, the masking agent includes a masking fluid that masks the one or more characteristic of the first detectable agent. In an exemplary embodiment, binding of an analyte of interest to the first detectable agent triggers, or initiates, the configuration of the masking agent to form the unmasked or mask-free-signal zone, which is indicative of the presence of the analyte. Formation of an unmasked or mask-free-signal zone may be detectable to the naked eye or may occur at a nanoscale. The unmasked or mask-free signal can include one or more of a visible or non-visible fluorescence, magnetic, mechanical, or physical signal.

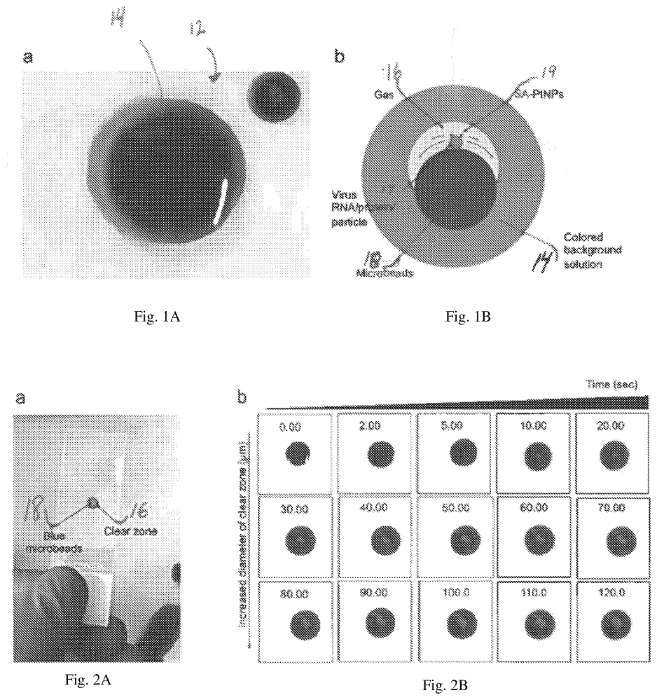

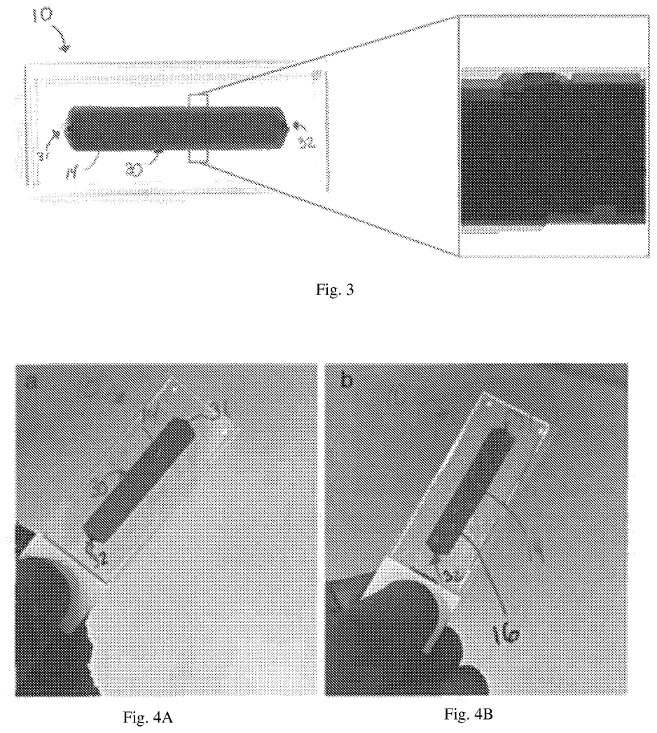

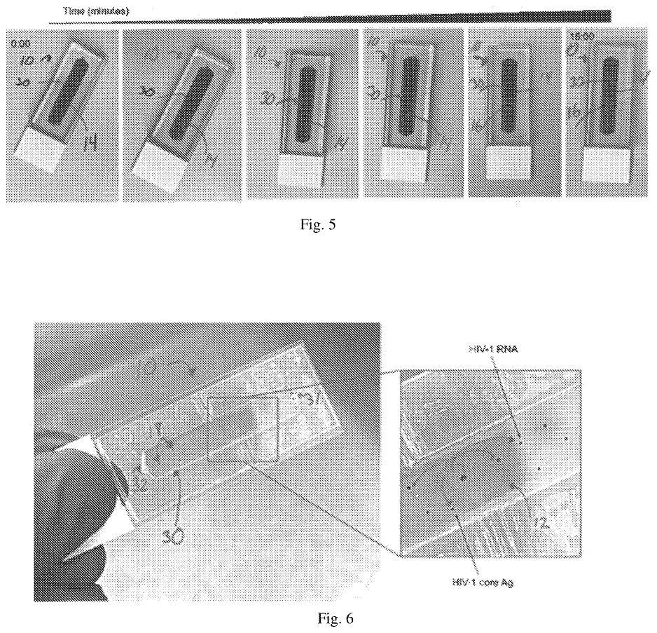

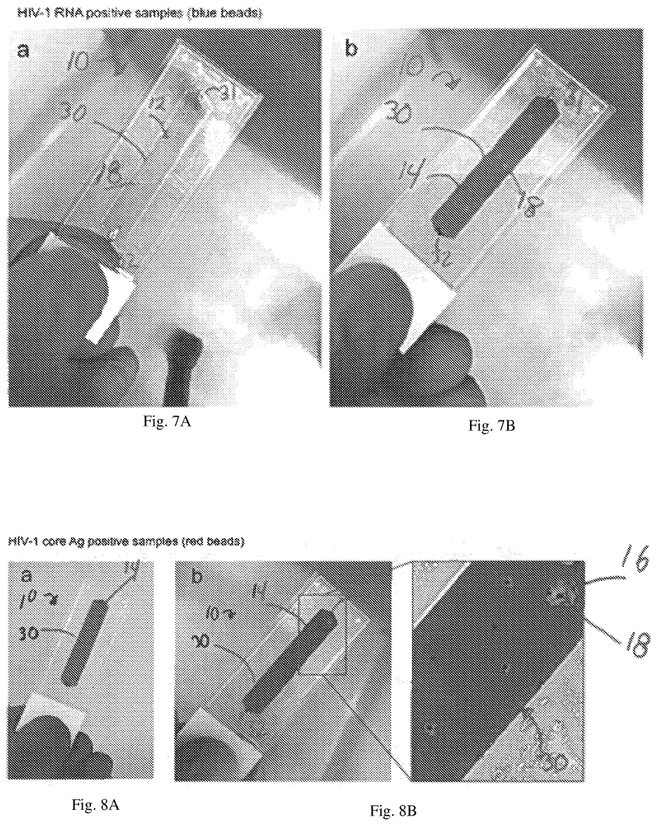

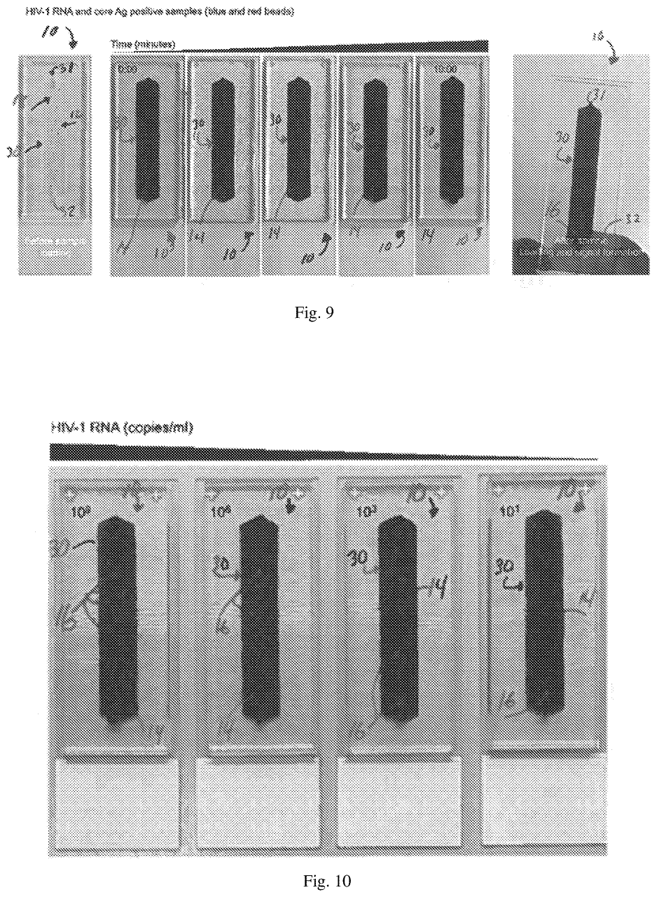

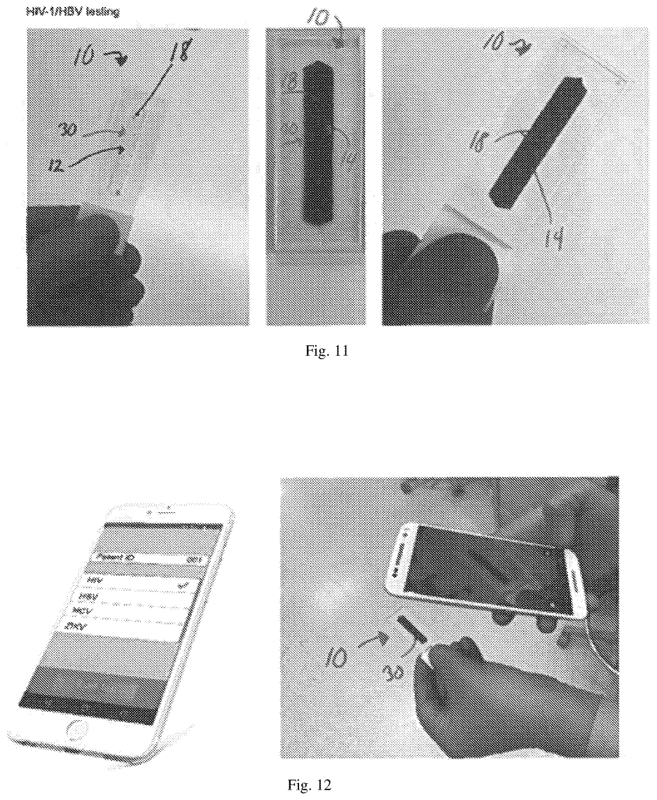

FIGS. 1-23 illustrates an example of an apparatus 10 for detection of an analyte in accordance with an aspect. Advantageously, the apparatus 10 may be configured as a point-of-care (POC) diagnostic microfluidic device and allow for immediate diagnostic feedback in contrast to remote laboratory testing typically required for analyte sensing approaches.

The apparatus 10 includes a substrate to which the first detectable agent is affixed or immobilized and a masking agent that masks the detection of the one or more characteristic of the first detectable agent in absence of the analyte. The at least one of the first detectable agent or the masking agent, such as a masking fluid 14, is configurable in the presence of the analyte to allow detection of the one or more characteristic of the first detectable agent which is indicative of the presence of the analyte. In some embodiments, binding of the analyte to the first detectable agent can disperse the masking agent, such as a masking fluid 14, around the first detectable agent to allow detection of the first detectable agent which is indicative of the presence of the analyte.

The first detectable agent may be added to the substrate either by printing or non-printing techniques on non-porous or porous substrates. In some embodiments, the first detectable agent can have a substantially circular, or spot shape on the surface of or within a substrate 12 and masked by the masking agent. The first detectable agent can also have a substantially star, triangle, square, rectangle, pentagonal, hexagonal, heptagonal, octagonal, etc. shape. The first detectable agent may also have a substantially alpha-numeric or another representative figure or symbol shaped. The first detectable agent, such as a spot shaped first agent, may be formulated from a functionalized material, (e.g., a biofunctionalized material) or non-functionalized material. Exemplary materials can include non-particulate material or particulate material including nanoparticles, microparticles, polymeric material, and non-polymeric material.

In some embodiments, the first detectable agent can include one or more first detectable particles 17 affixed to or immobilized on a surface of the substrate 12. For example, the one or more first detectable particles 17 can be adhered to, functionalized or chemically functionalized to and arranged on the surface of the substrate 12.

The first detectable particles 17 can be formed from an acrylate polymer. In an exemplary embodiment, the first detectable particles 17 can include colored poly(methyl methacrylate) (PMMA). The outer surface of each of the one or more first detectable particles 17 can also have a layer of a pegylated phospholipid. The pegylated phospholipid may cover about 1% to about 50%, about 5% to about 30%, about 10% to about 25%, about 18% to about 22%, or preferably about 20% of the outer surface of each of the one or more first detectable particles 17. The pegylated phospholipid can include, for example, 1,2 Disteraoyl-sn-glycero-3-phosphorylethanolamine (PEG-DSPE).

In some embodiments, the one or more first detectable particles can include a plurality of microbeads 18, such as colored PMMA microbeads. The microbeads 18 may have a diameter ranging from about 10 μm to about 1 mm. The one or more first detectable particles 17 can be configured to specifically bind to an analyte of interest.

In some embodiments, the first detectable particles 17 include a capturing agent on outer surfaces of the first detectable particles that specifically bind to the analyte. The capturing agent can include at least one of a small molecule, nucleotide, protein, antibody or streptavidin, or combinations thereof. In particular embodiments, the capturing agent can include an antibody capable of binding to the analyte. In certain embodiments, the capturing agent can include streptavidin.

In an exemplary embodiment, a capturing agent in the presence of an analyte of interest may adhere the analyte to the outer surface of one or more microbeads 18. For example, a capturing agent located on the surface of a microbead 18 may adhere the analyte to the microbead 18 outer surface when a fluid sample that includes the analyte of interest is loaded on the substrate of the apparatus 10.

The capturing agent may be directly conjugated to outer surfaces of the first detectable particle 17. The capturing agent may be functionalized to first detectable particles 17 covalently or non-covalently. In some embodiments, a linker can be used to provide covalent attachment of a capturing agent to the outer surface of a detectable particle 17.

The linker used to provide covalent attachment may be a homo-bifunctional linker or a hetero-bifunctional linker, depending upon the nature of the molecules to be conjugated. Homo-bifunctional linkers have two identical reactive groups. Hetero-bifunctional linkers have two different reactive groups. Various types of commercially available linkers are reactive with one or more of the following groups: primary amines, secondary amines, sulphydryls, carboxyls, carbonyls and carbohydrates. Examples of amine-specific linkers are bis(sulfosuccinimidyl) suberate, bis[2-(succinimidooxycarbonyloxy)ethyl]sulfone, disuccinimidyl suberate, disuccinimidyl tartarate, dimethyl adipimate 2HCl, dimethyl pimelimidate 2HCl, dimethyl suberimidate HCl, ethylene glycolbis-[succinimidyl-[succinate]], dithiolbis(succinimidyl propionate), and 3,3′-dithiobis(sulfosuccinimidylpropionate). Linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Linkers preferentially reactive with carbohydrates include azidobenzoyl hydrazine. Linkers preferentially reactive with carboxyl groups include 4-[p-azidosalicylamido]butylamine.

Heterobifunctional linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional linkers that react with carboxyl and amine groups include 1-ethyl-3-[3-dimethylaminopropyl]-carbodiimide hydrochloride. Heterobifunctional linkers that react with carbohydrates and sulfhydryls include 4-[N-maleimidomethyl]-cyclohexane-1-carboxyihydrazide HCl, 4-(4-N-maleimidophenyl)-butyric acid hydrazide.2HCl, and 3-[2-pyridyldithio]propionyl hydrazide.

In exemplary embodiments, the detectable agent or particle can be biofunctionalized with streptavidin (SA) to capture a biotin-modified nucleic acid target analyte (e.g., DNA and RNA), and/or biofunctionalized with an antibody to capture a protein target analyte (e.g., AMR enzyme, antigen and virus particles). By way of example, the biofunctionalization can be performed using a sequential conjugation protocol, which starts with activating the capturing agent with well-known N-Hydroxysuccinimide (NHS) chemistry, followed by adding crosslinking molecules of 3-(2-pyridyldithio)propionyl hydrazide (PDPH) or adipic acid dihydrazide (ADH). The PDPH-activated and ADH-activated capturing agent is then allowed to couple with thiolated SA molecules and oxidized antibody molecules, respectively. The surface 12 or at least one microchannel 30 can be optionally rinsed with PBS before processing samples.

Alternatively, the capturing agents may be non-covalently coated onto the outer surfaces of the first detectable particles 17. Non-covalent deposition of the capturing agents to the first detectable particles 17 may involve the use of a polymer matrix. The polymer may be naturally occurring or non-naturally occurring and may be of any type including, but not limited to, nucleic acid (e.g., DNA, RNA, PNA, LNA, and the like or mimics, derivatives or combinations thereof), amino acid (e.g., peptides, proteins (native or denatured), and the like or mimics, derivatives or combinations thereof), lipids, polysaccharides, and functionalized block copolymers. The capturing agent may be adsorbed onto and/or entrapped within the polymer matrix. Alternatively, the capturing agent may be covalently conjugated or crosslinked to the polymer, e.g., it may be “grafted” onto a functionalized polymer.

An example of a suitable peptide polymer for non-covalent deposition of the capturing agents to the first detectable particle 17 is poly-lysine, e.g., poly-L-lysine. Additional exemplary polymers can include block copolymers that comprise polyethylene glycol (PEG), polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(lactide-glycolide), copolyoxalates, polycaprolactones, polyesteramides, polyorthoesters, polyhydroxybutyric acid, polyanhydrides, poly(styrene-b-isobutylene-b-styrene) (SIBS) block copolymer, ethylene vinyl acetate, poly(meth)acrylic acid, polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, albumin and other hydrophilic proteins, zein and other prolamines and hydrophobic proteins, copolymers and mixtures thereof, and chemical derivatives thereof including substitutions and/or additions of chemical groups, for example, alkyl, alkylene, hydroxylations, oxidations, and other modifications routinely made by those skilled in the art.

An analyte detected using an apparatus 10 described herein can be a component of a biological fluid sample, such as a biomolecule found in a biological fluid sample obtained from a subject. Exemplary biomolecule analytes that may be detected include deoxyribonucleic acids (DNAs), ribonucleic acids (RNAs), proteins, polypeptides, peptides, polysaccharides, lipids, and the like. Further exemplary biomolecules include genes, gene fragments, messenger RNAs (mRNAs), hormones, vitamins, enzymes, coenzymes, immunoglobulins, and the like. In some embodiments, an analyte such as a peptide or nucleic acid of interest, can be modified prior to detection using the apparatus 10 in order to allow binding of the analyte to a capturing agent.

In an exemplary embodiment, where the capturing agent includes streptavidin, an analyte such as a peptide or nucleic acid of interest may be biotin modified prior to detection using the apparatus 10 in order to take advantage of the high affinity streptavidin has for biotin, thereby allowing binding of the analyte, or a complex such as an amplicon or multi-amplicon structure derived from the analyte to the capturing agent.

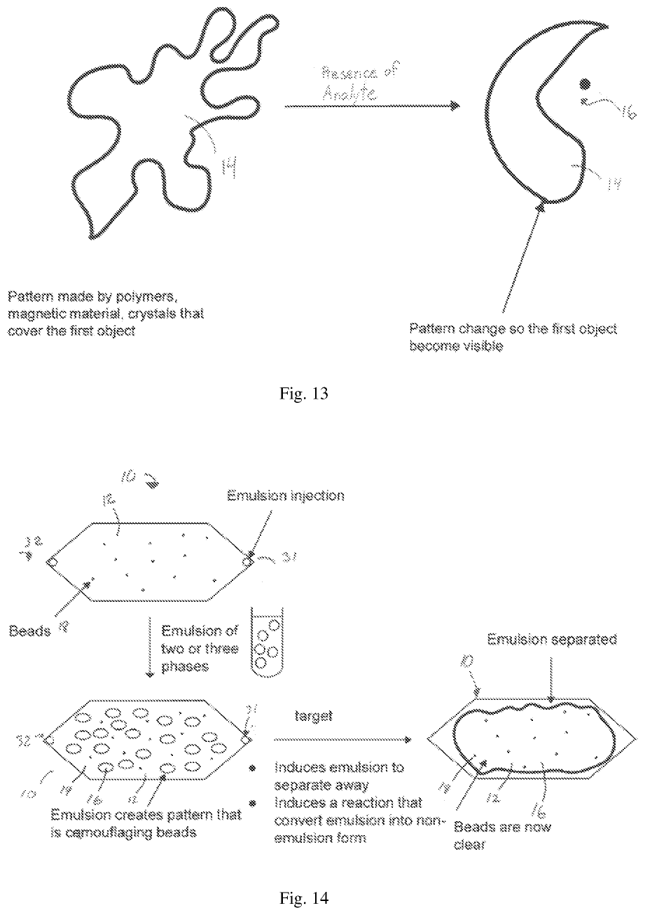

In some embodiments, the first detectable agent or particle may be masked, or camouflaged, using a patterned, patterned-like, or non-patterned presence of the masking agent. As shown in FIG. 13, in some embodiments, the masking agent can include polymers, magnetic material, or crystals that cover, mask, or camouflage the first detectable agent. In the presence of an analyte of interest, the patterned, patterned-like, or non-patterned character of the masking agent is changed, or altered, in a manner allowing for the first detectable agent to become detectable, e.g., through visual or non-visual detection. Visual detection of the first detectable agent can include the use of microscopes, imaging systems or simply the naked eye. Non-visual detection of the first agent can include fluorescent, ultraviolet (UV), and/or spectroscopic techniques depending on the composition of the first detectable agent.

In some embodiments, the masking or unmasking of the first detectable agent may be assisted through the use of an external or non-external force. Exemplary forces can include magnetic, photonic, mechanical, and chemical forces.

The masking agent can be catalytic, magnetic, photonic, polymeric, mechanical or in a gas form. In some embodiments, the masking agent that masks or camouflages the first detectable agent can include the biological on non-biological sample itself in which the analyte is to be detected within.

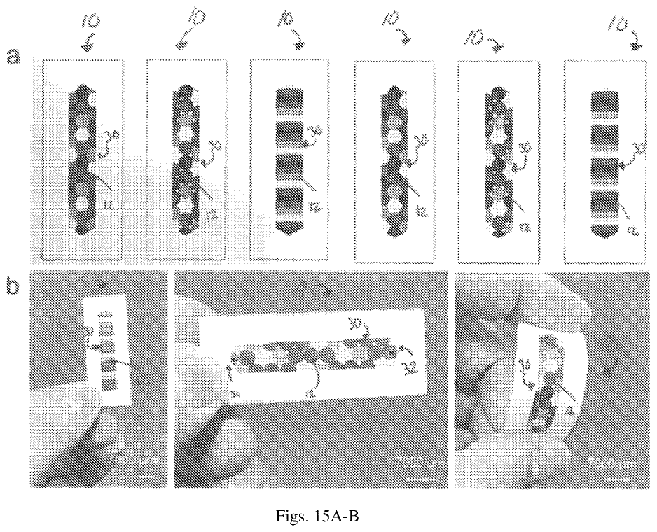

In other embodiments, the masking agent can cover, mask, or camouflage the first detectable agent where the first detectable agent is in a coded form and modification of a masking agent characteristic in response to the presence of an analyte allows for the code to be uncovered, unmasked or decamouflaged and the revealed code can then be deciphered. As shown in FIG. 15, the surface of a substrate 12 can be printed with various colored shapes and patterns where each color codes for a specific target analyte.

In some embodiments, the masking agent can include a masking fluid 14 that masks, or camouflages, the one or more characteristics of the first detectable agent. In some embodiments, the one or more first detectable particles 17 affixed to the surface of the substrate 12 are covered with a masking fluid 14 in an absence of the analyte.

In some embodiments, the masking fluid 14 may include a peroxide solution. For example, the peroxide solution may include about 1% to about 20% hydrogen peroxide (w/v), about 5% to about 15% hydrogen peroxide (w/v), and more preferably about 10% hydrogen peroxide (w/v). The masking fluid 14 may also include a surfactant and/or a degassing solvent. The masking fluid 14 may also be formulated as a viscous and/or a dense solution.

In some embodiments, the masking fluid 14 has a color that visually masks, or camouflages, the first detectable particle(s) appearance upon loading of the masking fluid 14 onto the surface of the substrate.

In other embodiments, the masking agent can include an emulsion of two or three phases. Two phase emulsion (i.e., water in oil (W/O) or oil in water (O/W)) has a dispersed phase and a continuous phase. Three phase emulsion (i.e., water in oil in water (W/O/W) or oil in water in oil (O/W/O)) has an inner phase and an outer phase (continuous phase) and dispersed phase. The emulsion can be prepared using a mechanical homogenizer and ultrasonic vibrator. The mechanical homogenizer uses a mechanical stirrer which rotates at a certain rpm to make the desired emulsion. As shown in FIG. 14, an emulsion of two or three phases is loaded onto a substrate having a number of optically visible microbeads 18 on the surface 12 such that the emulsion creates a pattern that mask, or camouflage, the microbeads 18. Upon introduction of a sample that includes a target analyte, the target induces a reaction that converts the emulsion into a non-emulsion form causing the emulsion to separate on the substrate surface 12. Separation of the emulsion unmask, or decamouflage, the microbeads allowing for visible detection of the microbeads 18 on the surface of the substrate 12.

In some embodiments, the apparatus 10 can further include a detection agent 19 that specifically binds to the analyte of interest to form a detection agent-analyte-particle complex. Binding of an analyte of interest to the first detectable agent and detection agent can occur sequentially or simultaneously. For example, the analyte may bind the first detectable agent before subsequently binding to the detection agent to form a first detectable agent-analyte-detection agent complex. Alternatively, the analyte may bind to the detection agent before binding to the first detectable agent to form a first detectable agent-analyte-detection agent complex. In other embodiments, the analyte can bind to both the first detectable agent and detection agent simultaneously or substantially simultaneously to form a first detectable agent-analyte-masking agent complex.

Simultaneous or sequential binding of the detection agent 19 and a detectable particle, such as a capturing agent on the outer surfaces of the first detectable particle, to the analyte can form a detection agent-analyte-particle complex. In certain embodiments, the detection agent may be masked, e.g., camouflaged, by the masking fluid 14 when the analyte is not bound to both the detectable particle and the detection agent. For example, the masking fluid 14 can visually mask detectable particles 17 that do not form a detection agent-analyte-particle complex.

In some embodiments, the detection agent 19 of the complex catalyzes degradation of the masking fluid 14 into a gas that physically disperses masking fluid 14 around the complex. Such dispersal from catalytic conversion of the masking fluid 14 can form a fluid-free, unmasked signal zone, or mask-free zone 16, which allows for optical detection of the complex on the substrate, thereby indicating the presence of the analyte. For example, the detection agent can include a platinum nanoparticle (PtNPs) that target or specifically bind to the analyte or analyte bound to the first detectable agent or particle. PtNPs can be conjugated to a targeting moiety, such as, but not limited to a streptavidin, aptamer, or antibody targeting moiety. PtNPs bound to the analyte vial the targeting moiety can catalyze degradation of peroxide masking fluid into a gas that physically disperses the masking fluid.

The substrate of the apparatus 10 to which the detectable agents or particles are affixed may be configured to hold a specific volume of sample in order to maximize testing sensitivity and overall performance of the apparatus 10. The term, “substrate” as used herein refers to surfaces as well as solid phases. In some cases, the substrate is non-porous and may include glass, plastic, metal, or a non-porous polymer substrate material. A substrate may also include components including, but not limited to a poly-dimethylsiloxane (PDMS), silicon, or quartz. A substrate can also be a non-porous substrate, such as, but not limited to paper, wood, or a porous polymer substrate material.

In some embodiments, the substate can include one or more microchannels 30. The microchannel 30 may extend along at least a portion of the substrate. The microchannel 30 may then include or house the first detectable particles and the masking fluid. The substrate can include a substantially planar transparent wall that defines a surface of at least one of the microchannels 30. This substantially planar transparent wall, which can be, for example, glass or plastic, permits observation into the microchannel 30 by the naked eye or an imaging system to detect the one or more characteristic of the first detectable agent or the masking agent which is indicative of the presence of the analyte.

In some embodiments, the microchannel(s) 30 can have a depth or height of 0.5 μm to 1 mm, 10 μm to 500 μm, 100 μm to 400 μm, or 325 μm to 375 μm. The microchannel(s) 30 can have a depth or height of up to 0.5 μm, 1 μm, 10 μm, 100 μm, 200 μm, 300 μm or more. In an exemplary embodiment, the microchannel(s) 30 have a depth or height of about 340 μm.

In some embodiments, the at least one microchannel 30 can have a width of 100 μm to 15 mm, 1 mm to 10 mm, or 6 mm to 8 mm. In an exemplary embodiment, the at least one microchannel 30 can have a width of about 7 mm. In some embodiments, the at least one microchannel 30 can have a cross-sectional area, perpendicular to the flow direction of a loaded fluid sample, of 500 μm², 1000 μm², 10,000 μm², 20,000 μm², 30,000 μm 2 or more.

The microchannel 30 may include a fluid inlet 31 for receiving fluids, such as a fluid sample which may include an analyte of interest. The microchannel 30 can further include an outlet 32 for removing fluid, e.g., a fluid sample, from the microchannel 30 after the fluid has been in the presence of the first detectable agent and/or masking agent. In some embodiments, the fluid is removed via the fluid outlet 32 after an amount of time sufficient to allow binding of the analyte of interest in the fluid to the first detectable agent and/or masking agent.

In some embodiments, the substrate can include a multilayer structure formed of a base layer, a microchannel 30, and a cover layer. The multilayer structure may further include intermediate layers. In some embodiments, a first end of the microchannel 30 may be aligned with a corresponding fluid inlet 31 port and a second end of the microchannel 30 is aligned with a corresponding fluid outlet 32 port. This creates a flow channel from a fluid inlet 31 port to the corresponding fluid outlet 32 port via the microchannel 30. The microchannel 30 can also extend slightly beyond its respective fluid inlet 31 port and fluid outlet port 32. The microchannel 30 is sized to accept volumes, e.g., μL or mL, of the fluid sample containing sufficient target analyte in the presence of the first detectable agent and/or masking agent to allow detection of the analyte and/or maximize testing sensitivity.

The base layer of the substrate can provide structural support to the microchannel and is formed of a sufficiently rigid, optically transparent, and gas impermeable material, such as poly(methyl methacrylate) (PMMA) or glass. The base layer can have a suitable thickness, for example of about 0.1 mm to about 2 mm, or about 0.6 to about 1.6 mm, as determined by manufacturing and assembly restrictions.

The cover layer may contain the fluid inlet 31 ports and fluid outlet 32 ports used to feed the sample in/out of the microchannel 30. The cover layer thickness can be about 1 mm to about 10 mm, for example, about 3.175 mm, and is determined by the integration and assembly requirements. The fluid inlet 31 and fluid outlet port 32 diameters can be about 0.3 mm to about 3 mm, for example about 1 mm. The lower size limit is determined by the manufacturing restrictions. The upper size limit is determined by the load conditions of the sample into the microchannel 30. In an exemplary embodiment, a laser cutter can be used to cut a larger piece of PMMA into a desired size for the apparatus 10 (e.g., a microfluidic device) and/or to cut holes for the fluid inlet 31 ports and the fluid outlet 32 ports.

The intermediate layers can be adhered to the base layer around the microchannel 30 after the microchannel 30 is placed on the base layer. The cover layer, which can have substantially the same lateral dimensions as the base layer and any intermediate layers, can be adhered onto the exposed side of the intermediate layer, thereby enclosing the microchannel 30. In an exemplary embodiment, the cover layer may be attached to the base layer using a double-sided adhesive (DSA). In preferred embodiments, the apparatus 10 is oriented such that the cover layer is on top. Alternatively, the apparatus 10 can be oriented such that the cover layer is on the bottom.

In some embodiments, the surface of a substrate 12 can be functionalized with a plurality of first detectable particles, which include a capturing agent on outer surfaces of the first detectable particles, that specifically bind to the analyte to capture or adhere the analyte when a sample fluid containing the analyte is loaded onto the surface of the substrate 12 or passed through or perfused through at least one microchannel 30 of the substrate. If the apparatus 10 includes multiple microchannels 30, each microchannel 30 can be functionalized with detectable particles having a capturing agent capable of adhering different analytes of interest thereto, or even different epitopes of an individual analyte of interest thereto. In any case, each microchannel 30 is configured to receive and provide analyte detection analysis of a microvolume fluid sample.

Optionally, the apparatus 10 for the detection of an analyte can further include one or more second detectable agent. In some embodiments the second detectable agent can include second detectable particles affixed to the surface of the substrate 12. The second detectable particles can include a second plurality of second capture agents to specifically bind a second analyte. The apparatus 10 can further include a plurality of second detection agents including a second targeting moiety to specifically bind the second analyte. In such cases, binding of the second analyte to both the second detectable particles and the second detection agent forms a second detection agent-analyte-particle complex that forms a fluid-free zone 16 around the second agent-analyte-particle complex allowing for detection of the second agent-analyte-particle complex allowing for detection of the second agent-analyte complex on the substrate. In some embodiments, the one or more second detectable particle(s) are a substantially different color than the first detectable particle(s).

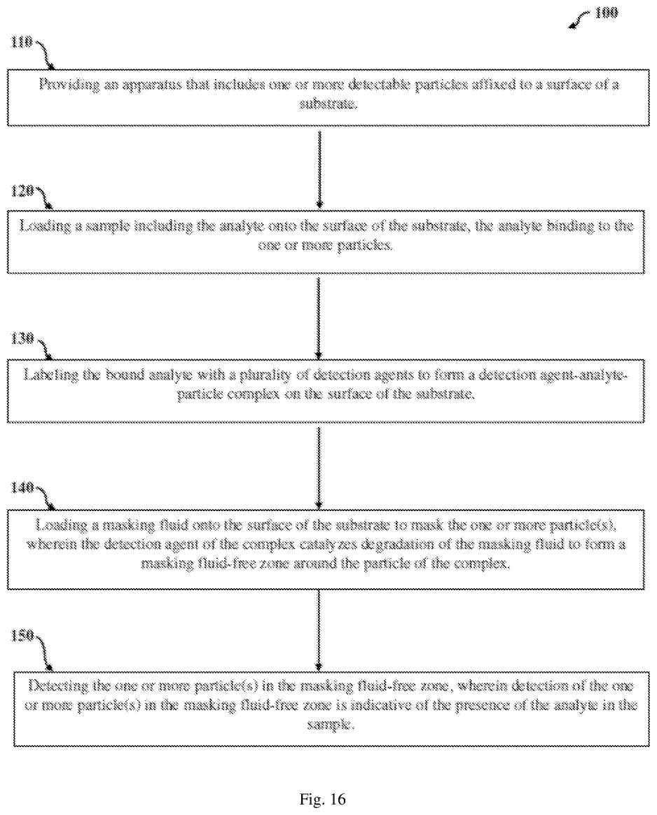

Other embodiments described herein relate to a method 100 (FIG. 16) for detecting an analyte in a sample. In one example, the method 100 can be performed using an apparatus 10 illustrated in FIGS. 1-23 and described above. The method 100 can find use in a variety of settings and with a number of applications, such as in a point-care environment or for high-throughput analysis.

At Step 110 of the method 100, an apparatus is provided. The apparatus 10 as described above, generally includes one or more detectable particles affixed to a surface of a a substrate.

At Step 120, a sample including the analyte is loaded on to the substrate surface 12 where the analyte binds to the one or more particles. The sample loaded on to the surface of the substrate 12 may include two or more different analytes and the apparatus 10 can be configured to detect two or more different analytes from the sample.

At Step 130 of the method 100, the bound analyte is labeled with a plurality of detection agents to form a detection agent-analyte-particle complex. In some embodiments, the particles of the detection agent-analyte-particle complex can include a microbead 18 affixed to the surface of the substrate 12, thereby forming a detection agent-analyte-microbead complex on the surface of the substrate 12.

At Step 140 of the method 100, a masking fluid 14 is then loaded onto the surface of the substrate 12 to mask, or camouflage, the one or more first detectable particles of the complex. The first detection agent of the first detection agent-analyte-first detectable particle complex on the surface of the substrate 12 may then catalyze conversion, e.g., through catalytic degradation, of the masking fluid to form a masking fluid-free zone around the one or more first detectable particles 17 of the complex.

At Step 150 of the method 100, the one or more first detectable particles 17 in the masking fluid-free zone 16 around the particle complex are detected. Detection of the one or more first detectable particles 17 on the surface of the substrate 12 is indicative of the presence of the analyte in the sample.

Detection of the one or more first detectable particles 17 of the complex may include optically detecting the one or more first detectable particles 17 on the surface of the substrate 12 by imaging the surface of the substrate. Optically detecting the one or more first detectable particles 17 can include the use of any suitable imaging device capable of obtaining an image of the substrate surface. In some embodiments, imaging the surface of the substrate 12 is performed using a camera, such as a camera included as part of a mobile device (e.g., mobile phone, see FIG. 12). The method 100 may then further include determining the presence of an optically first detectable particle in at least one image obtained from the masking fluid-free zone on the surface of the substrate. In some embodiments, the number of first detectable particle(s) and/or the size of a masking fluid-free zone(s) optically detected can be indicative of the amount and/or concentration of a target analyte in the sample.

The method 100 of detecting an analyte in a sample can further include a washing step to remove uncaptured analyte and the remaining sample from the surface of the substrate 12 prior to labeling the bound analyte with the plurality of first detection agents. An additional washing step may be performed to remove unbound first detection agents from the surface of the substrate after allowing a suitable amount of time for binding of the first detection agent to the analyte-first detectable particle complex.

In an exemplary embodiment, the analyte to be detected using the method 100 is a nucleic acid, and the method 100 further includes the step of contacting the nucleic acid analyte in the sample with one or more biotin-modified nucleic acid primers specific for the nucleic acid analyte to form a biotinylated nucleic acid target analyte. The biotinylated nucleic acid target analyte is then bound to the outer surface of the one or more first detectable particle(s) 17 on the surface of the apparatus 10 substrate by streptavidin conjugated to the particle(s).

The biotin modified nucleic acid primers can include a set of biotin-modified nucleic acid primers specific for the nucleic acid analyte. In an exemplary embodiment, the set of biotin modified nucleic acid primers can target different sequences in the nucleic acid analyte. In other embodiments, the set of biotin modified nucleic acid primers can target overlapping sequences in the nucleic acid analyte.

In some embodiments, the biotin modified nucleic acid primers can include a multi-arm nucleic acid primer. A multi-arm nucleic acid primer for use in a method or system described herein can include sense and anti-sense arms of single stranded oligonucleotides that are linked to a core by a flexible linker. The anti-sense arms(s) complement an anti-sense sequence of a target nucleic acid (e.g., DNA or RNA) and the sense arm(s) complement(s) a sense sequence of the target nucleic acid analyte (e.g., DNA or RNA) distinct from and subsequent to the anti-sense sequence.

In some embodiments, the multi-arm nucleic acid primer can include a polyethylene glycol (PEG) linker to link the sense arm(s) to the anti-sense arm(s) thereby forming a multi-arm PEG-nucleic acid primer. In some embodiments, the multi-arm primer includes two, three, four, five or more sense arms and two, three, four, five or more anti-sense arms. In certain embodiments, the multi-arm primer includes at least two sense arms and at least two anti-sense arms. In addition, one or more arms are terminally modified with biotin.

The sense arms and the anti-sense arms can be conjugated to the multi-arm PEG linker using click chemistry, such as maleimide chemistry. The sense arms and the anti-sense arms can be configured to bind to the complementary sequence in the target nucleic acid and form looped-arm double sided (ds) nucleic acid (e.g., dsDNA) structure mediated by the sense arms. In other embodiments, the anti-sense arms can prime for amplification, followed by self-priming with sense arms generating a stem-looped nucleic acid hybrid structure in which loops are partially made of the sense arm and anti-sense arm and the linker. Other free anti-sense arms and sense arms of the multi-arm primer can mediate a chain of self-looping, leading to the formation of multi-arm amplicons. The multi-arm amplicons can then be used as templates in generating large multi-amplicon structures and self-prime for the formation of new strand and stem-looped structures. The self-looping can continue until all the sense arms expand into a copy of the target analyte sequence.

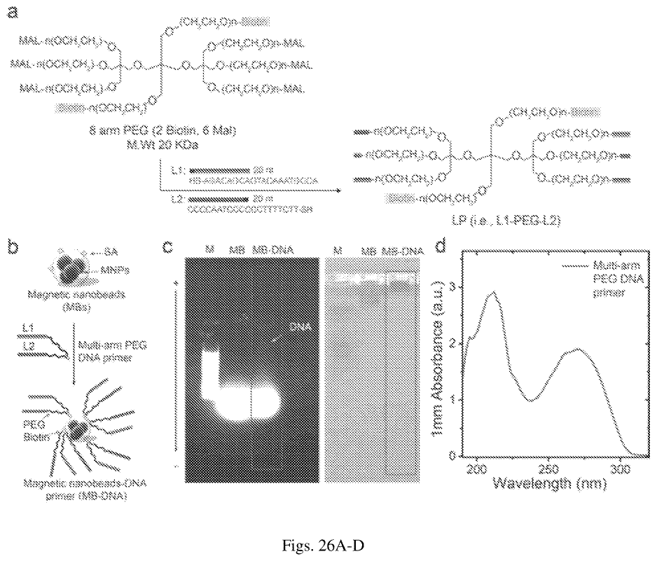

In an exemplary embodiment, as shown in FIG. 26(A) the multi-arm nucleic acid primer can include branched PEG molecules with eight side chains, where each side chain serves as an arm of the primer. Each individual arm of the multi-arm primer can be functionally modified to facilitate capture of the primer by a capture agent, (e.g., a particle conjugated to streptavidin) or to facilitate coupling of a nucleic acid primer sequence. For example, as further shown in FIG. 26(A), two of the eight side chains are terminally modified with biotin and the remaining six are maleimide-modified chains coupled to a copy of a single-stranded (ss) nucleic acid that either complements the sense or anti-sense sequence of the target nucleic acid to form a multi-arm looping primer. In particular embodiments, the copies of single stranded nucleic acid in the multi-arm PEG-nucleic acid primer are 50:50% sense to anti-sense sequences.

In some embodiments, the use of a multi-arm primer, for example prior to detection using an apparatus 10 and/or method 100 described herein, allows for a rapid amplification (i.e., a multi-arm primer-based amplification (MAPA) reaction method) of an analyte in a sample with high efficiency under isothermal conditions.

As illustrated in FIG. 25, a MAPA reaction method can include the use of a unique set of three nucleic acid primers that target a total of four distinct sequences in the target nucleic acid analyte. The three nucleic acid primers for use in a MAPA reaction include an initiator primer (IP) and an unlooping primer (ULP) that complement sequences at the start and the end of the target nucleic acid analyte, respectively, in addition to a multi-arm PEG-nucleic acid looping primer. The multi-arm nucleic acid looping primer (LP) includes looping sequence 1 (LP1) and looping sequence 2 (LP2) that are designed with anti-sense and sense nucleic acid oligonucleotides that specifically complement two distinct but subsequent sequences in the target nucleic acid, designated L1 and L2. In exemplary embodiments, LP1 and LP2 are each about 20-mer in length and can be thiolated on their 3′ and 5′, respectively.

As further illustrated in FIG. 25, a MAPA reaction can start with the anti-sense arms of the multi-arm PEG-nucleic primer binding, or annealing, to the complementary sequence in the target nucleic acid, followed by the formation of looped-arm double sided (ds) nucleic acid structures (e.g., dsDNA or dsRNA) mediated by the arms carrying the sense sequences. This formation serves as a template for nucleic acid synthesis via continuous cycles of alternating looping and unlooping steps and the formation of multi-arm amplicons that are consumed in an elongation step of the MAPA reaction, thereby forming large multi-amplicon nucleic acid structures. The large multi-amplicon nucleic structures developed using a MAPA reaction can be developed in <30 minutes and allow for the rapid and specific detection of nucleic acid analytes using an apparatus 10 and/or method 100 described herein. In an exemplary embodiment, multi-arm amplicon nucleic acid structures developed using a MAPA reaction allow for the specific detection of human immunodeficiency virus-1 (HIV-1) with a sensitivity down to about 10³ copies/ml. In some embodiments, a MAPA reaction can be used to amplify a target nucleic acid analyte to form biotinylated MAPA amplicons that can be spontaneously captured on streptavidin conjugated microbeads (see e.g., FIG. 30), such as those on the surface of a substrate 12 of an apparatus 10.

In some embodiments, multi-arm PEG-nucleic acid primers can be prepared with oligonucleotides that target different nucleic acid sequences. Such multi-arm nucleic acid primers can be used for amplifying different analyte targets in a sample or even different epitopes on same target analyte at the same time using a MAPA reaction amplification method.

In other embodiments, an alternative nucleic acid amplification method can be used to amplify a target nucleic acid analyte in a sample prior to detection using an apparatus 10 and/or method 100 described herein. An alternative nucleic acid amplification method can include a polymerase chain reaction (PCR), or another isothermal amplification method besides a MAPA reaction amplification method described herein. For example, a variety of isothermal nucleic acid amplification methods have been developed to avoid the added components required of thermal cycling required by PCR. An alternative isothermal amplification technique may be used with the invention can include, but is not limited to, Nucleic acid sequence-based amplification (NASBA), transcription mediated amplification (TMA), self-sustained sequence replication (3 SR), signal-mediated amplification of RNA technology (SMART), strand displacement amplification (SDA), rolling circle amplification (RCA), loop-mediated isothermal amplification of DNA (LAMP), isothermal multiple displacement amplification (TMDA), helicase dependent amplification (HDA), single primer isothermal amplification (SPIA), circular helicase-dependent amplification (cHDA), and recombinase-polymerase amplification (RPA).

An isothermal nucleic amplification method can include the use of a nucleic acid polymerase with high strand displacement activity in order to enable nucleic amplification at a constant temperature (i.e., isothermal amplification). Additional reagents for use in an isothermal amplification method can include deoxynucleoside triphosphates (dNTPs) and suitable buffers. In a further embodiment for amplifying RNA target biomolecule analytes reagents for use in isothermal amplification can include a reverse transcriptase. Typically, a method of amplifying a nucleic acid target analyte using an isothermal amplification method includes providing a primer, such as a multi-arm primer described herein, and annealing the primer to the target nucleic acid analyte (e.g., DNA or RNA). After annealing to the nucleic acid target sequence, the primer can be extended using a polymerase having strand displacement activity to form a nucleic acid template.

Exemplary high strand displacement activity nucleic acid polymerase for use in an isothermal nucleic acid amplification method can include both naturally and artificially derived nucleic acid polymerases. Exemplary high strand displacement activity polymerases can include, but are not limited to, natural polymerases such as Bst, BSU, and Phi29, as well as artificial polymerases, such as the heat resistant or thermostable DNA polymerase SD polymerase. In a particular embodiment, the high displacement activity nucleic acid polymerase for use in an isothermal nucleic amplification method described herein prior to detection is Bst. The Bst DNA polymerase or Bacillus stearothermophilus DNA Polymerase I is a typical member of polymerase family A and its structure is similar to the structure of Taq DNA polymerase or other members of the family.

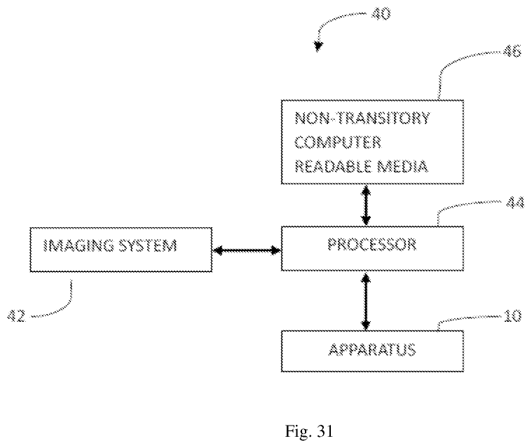

Still other embodiments described herein relate to a system 40 (see FIG. 31) including an apparatus 10 described above, an imaging system 42 (e.g., a camera), a processor 44 (e.g., a microprocessor), and one or more non-transitory computer readable media 46 to store executable instructions and data. The system 40 allows the microprocessor 44 to access the media 46 and execute the instructions to perform a method of detecting the analyte, preferably, according to a method 100 described herein. The executable instructions may include a camera interface configured to instruct the camera to capture at least one image, e.g., an image of the surface substrate 12 of an apparatus 10.

A system including an apparatus 10 described above can also include an imaging system 42 for optically detecting the one or more characteristic of the first detectable agent or the masking agent which is indicative of the presence of the analyte after the fluid sample containing the analyte is loaded onto the surface of the substrate 12.

In some embodiment, the imaging system 42 can detect unmasked or decamouflaged particles(s) in the masking fluid-free zone 16 on the surface of the substrate 12 after the fluid sample containing the analyte is loaded onto the surface of the substrate 12. The imaging system 42 can be a lens-based imaging system, lens less imaging system, and/or mobile imaging system, e.g., cellular phone camera. The imaging system 42 can include a control unit, which can include a computer readable storage unit and a processor to analyze the images of the surface of the substrate 12 and provide real-time feedback to a subject of the results of the image acquisition/analysis. These results, in turn, can be readily transmitted to a primary care provider and/or stored in a medical record database.

In some examples, the imaging system 42 can be a lens-based imaging system or a lens less/mobile imaging system. In some embodiments, the lens less imaging system can be a CCD sensor and a light emitting diode. In some embodiments, the lens less imaging system can include a fluorescent microscopy camera and fluorescent motorized microscope with imaging and analysis software to obtain real-time microscopic images. In some embodiments, video images obtained can be converted to single frame images for further processing and analysis. In some embodiments, the images can then be analyzed by using Adobe Photoshop software (San Jose, CA).

In some examples, a mobile imaging and quantification algorithm can be integrated into, or with, the apparatus 10. The algorithm can achieve reliable and repeatable test results for data collected in all resource settings of the apparatus 10.

In some embodiments, the apparatus 10 can be configured as a microfluidic device to cooperate with a mobile device, such as a cellular phone having imaging capabilities. In such a case, the cellular phone can be provided with or capable of obtaining image analysis algorithms/software, e.g., via an online application. Images can be recreated by the cellular phone camera software and loaded into a custom phone application that identifies one or more characteristic properties of a first detectable agent or a masking agent which is indicative of the presence of the analyte, such as one or more first detectable particle(s) 17 in the masking fluid-free zone 16, in the image, and displays the results.

In some embodiments, images obtained from an imaging system 42 can be sent to a control unit that includes a computer readable storage medium 46 for storing executable instructions and data, such as the images, and a microprocessor 44 to access the media that and execute instructions for capturing images. The images can be correlated the presence of an analyte using the processor 44 or another processor.

The image processing may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, a computer readable medium 46 (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) can be encoded with one or more programs that, when executed on one or more computers or other processors, perform methods that implement the various embodiments described herein. The computer readable medium or media 46 can be transportable, such that the program or programs stored thereon can be loaded onto one or more different computers or other processors to implement various aspects described herein. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects as discussed above. Additionally, it should be appreciated that according to one aspect of this embodiment, one or more computer programs that when executed perform methods of described herein need not reside on a single computer or processor but may be distributed in a modular fashion amongst a number of different computers or processors to implement various aspects herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Any appropriate analyte may be evaluated using a method 100, system 40, and/or apparatus 10 described herein, typically provided that a sample may be obtained that contains the analyte of interest. The sample can be obtained directly or indirectly by acquiring a biological sample from a subject. For example, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), saliva, cerebrospinal fluid (CSF), bile, bone marrow aspirate, breast milk, plasma, serum, sputum, stool, synovial fluid, urine and oral, nasal and/or vaginal fluids from one or more subjects.

In further embodiments, the presence and/or the amount of an analyte detected in a biological sample using the system 40, method 100, or apparatus 10 described herein can be compared to a standard or control to indicate whether the subject has the condition or disease; and optionally, diagnosing the subject as having the condition or disease based on the results. The appropriate standard or control can be the amount of analyte detected using the apparatus 10 that was obtained from a subject who is identified as not having the condition or disease.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

### Example 1

We developed a new type of camouflaging-based microfluidic (CamoChip) apparatus and system for POC diagnostics that enables analyte load testing in minutes. The apparatus and system integrate microfluidics, nanotechnology, surface chemistry, and AI image analysis to create bio-inspired detection of the target biomarkers on a chip. We use a microchip loaded with colored beads, functionalized with target-specific molecules (i.e., antibody or oligonucleotides), that are embedded in a peroxide fluid of similar color to mask their color. In the absence of the target biomarker, beads remain masked with the color of the fluid (i.e., camouflaged—visually invisible), and in the presence of the target biomarker, it triggers the rapid accumulation of PtNPs on the surface of the beads, which catalyzes the conversion of the surrounding fluid into gas, unmasking the beads by forming a color-free area around the beads (i.e., decamouflaged—visually visible). Beads are 300 m in size for easy visual detection with simple optical systems, such as a cellphone and come in one or multiple colors for easy multiplex testing. In this Example, HIV-1 RNA is complexed with a set of DNA primers that target multiple conserved HIV-1 sequences (e.g., HIV-1 pol-integrase and LTR genes), and terminally modified with biotin and digoxigenin. The formed complexes with biotin and digoxigenin are captured on the surface of dark blue beads modified with anti-biotin monoclonal antibody (anti-biotin mAb) and labeled with PtNPs modified with anti-digoxigenin monoclonal antibody (anti-digoxigenin mAb). The surface of the beads is additionally engineered with a novel surface chemistry that uses a pegylated phospholipid polymer of 1,2-Distearoyl-sn-glycero-3-phosphorylethanolamine (DSPE). The hydrophobic properties of the DSPE facilitate rapid repelling of the blue-colored liquid away from the surface of beads, which is necessary to promote the effective accumulation of O₂ gas around the bead as a clear zone to unmask the beads. The CamoChip approach provides a novel system for non-amplification-based VL testing (a key challenge for effective testing and monitoring at the POC) without the need for staining or multistep labeling. The use of the beads' array supports high sensitivity and specificity coupled with the simplicity of colorimetric assays to form a revolutionary test for HIV RNA at POC.

Our approach is fundamentally different from all existing techniques, allowing rapid visual detection of multiple targets in a rapid, accurate, low cost and simple way. The localized accumulation of the gas signal, resulting in variable sized clear areas around the beads, makes the optical detection of the target biomarker molecules more sensitive than other sensing approaches that utilize color (e.g., ELISA) and fluorescence (e.g., PCR) labels. With current approaches, the generated color or fluorescence signals generally are diffused over a relatively large volume of sample that leads to lower detection sensitivity and sensing efficiency. Furthermore, the color-coded beads nature of this approach enables the easy and efficient integration of the chip with AI-based image analysis systems. This supports a powerful universal modality that can be adapted to simple camera-driven optical systems. Such advancements are a major step towards true POC and self-diagnostics.

### Experiments and Results

### CamoChip Design and Development

CamoChip requires two major components for sample testing: (i) a microchip loaded with surface-functionalized colored beads (i.e., with antibody or DNA) to capture the target molecules onto the chip, and (ii) Pt-nanoprobes prepared of catalytically active PtNPs surface functionalized with target specific molecules (i.e., antibody or DNA) to label the captured target on the bead and form the clear area. The two components, in the presence of target molecules, form a three-component complex of PtNP-Target-beads on the surface of the chip. We follow a working protocol incorporating the following steps to allow highly specific formation of this complex on the chip: (i) sample loading on chip and target capture on the surface of antibody-modified colored beads, (ii) target labeling with antibody-functionalized PtNPs (i.e., Pt-nanoprobes) on chip and (iii) loading the chip (with the PtNP-target-bead complex) with a colored peroxide solution. In the presence of a target, Pt-nanoprobes on the surface of beads catalyze the conversion of the colored peroxide solution, which is masking the beads, releasing O₂ gas that accumulates around the beads, pushing the fluid away, unmasking the beads allowing them to be easily visualized and detected by a portable, low-cost digital platform.

Following this design and working protocol, we used human immunodeficiency virus (HIV)-1 as a model clinical target to develop and optimize CamoChip with two color beads, blue and red, coded for HIV-1 RNA and HIV-1 p24 capsid protein.

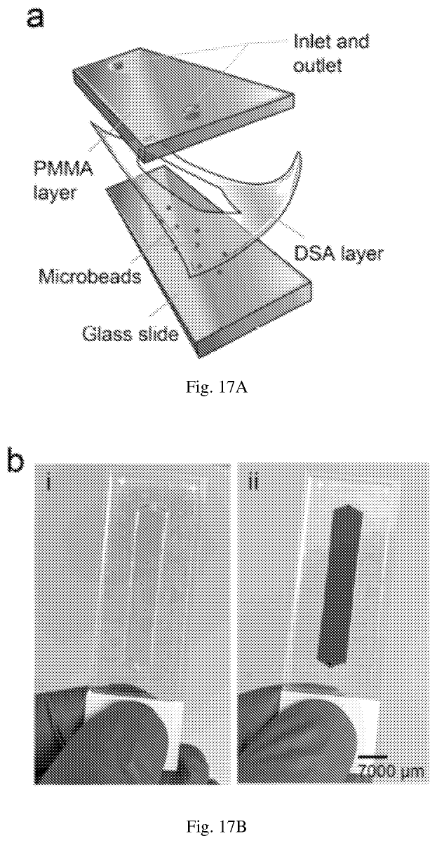

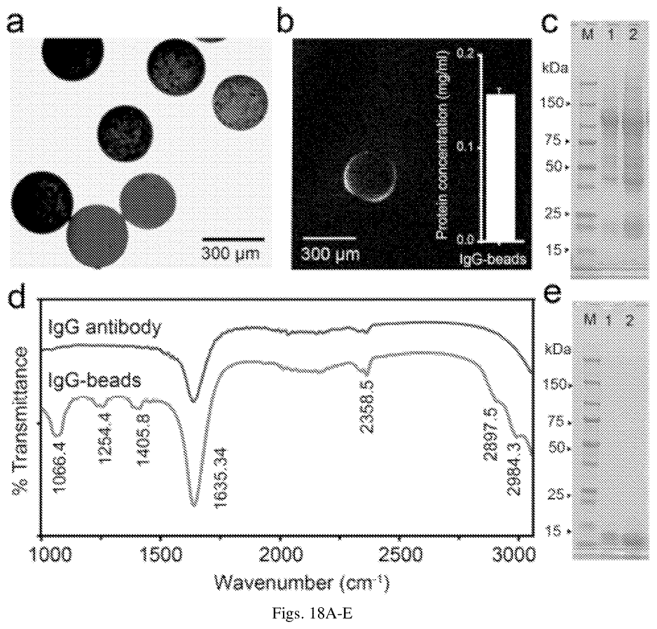

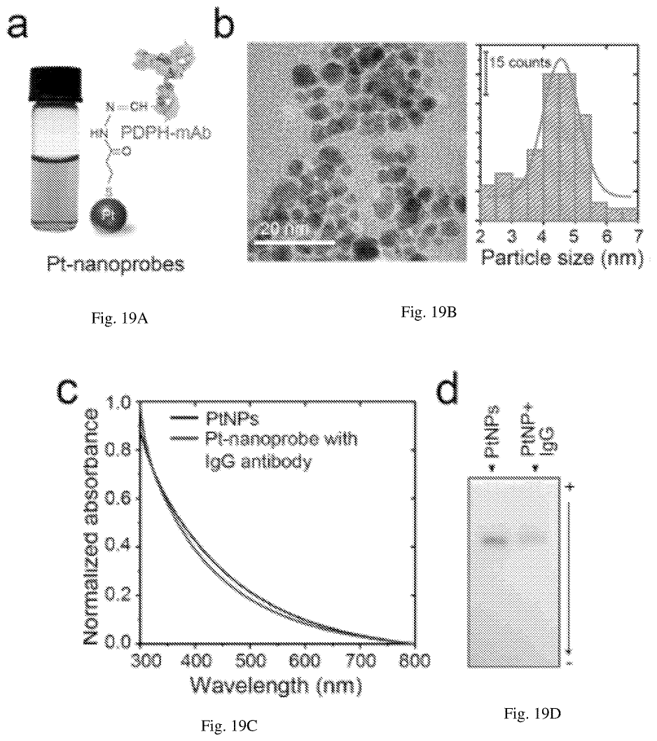

To prepare the microchip, a 3 mm laser-machined poly(methyl methacrylate) (PMMA) (3.175 mm; McMaster-Carr, Inc.) assembled on a glass substrate (25×75 mm; Globe Scientific, Inc.) using a double layer of double-sided adhesive (DSA) (170.18 μm; 300LSE, 3M, Inc.) to form a single microfluidic channel of 7 mm in width and 340 μm in depth. The glass surface of the microchip was initially assembled with the DSA layers then the microfluidic channel was modified with a layer of liquid plastic (200 μm in thickness), in which biofunctionalized colored microbeads were embedded, with 200 μm in the plastic layer and 100 μm as free surface for target capture (FIG. 17). The PMMA layer was added on the top of DSA to form an inlet and outlet of the microfluidic channel. The beads' color is used to encode for a specific hapten molecule, and beads of the same color are all surface functionalized with the same target-specific ligand to allow highly efficient capture of the target. The surface of the blue beads was functionalized with streptavidin (SA) to capture the biotin-modified HIV-1 RNA, while the surface of the red beads was functionalized with anti-HIV-1 p24 monoclonal antibody to capture p24. The carboxylated surface of the beads was biofunctionalized using a sequential conjugation protocol, which starts with activating the surface of the beads with the well-known NHS chemistry, followed by adding crosslinking molecules of 3-(2-pyridyldithio)propionyl hydrazide (PDPH) on blue beads or adipic acid dihydrazide (ADH) on red beads. The PDPH-activated and ADH-activated beads are then allowed to couple with thiolated SA molecules (Nanocs, Inc.) and oxidized HIV-1 p24 antibody molecules (Abcam, Inc.), respectively. The modification of the surface of different beads was confirmed using UV-vis, fluorescence spectroscopy, FT-IR and sodium dodecyl sulfate-polyacrylamide gel electrophoresis (SDS-PAGE) techniques (FIG. 18). UV-vis absorbance confirmed the conjugation of p24 antibody with an average concentration of 0.16±0.01 mg/ml to the surface of beads (FIG. 18B). After staining the surface of beads with FITC-modified G protein, fluorescence microscopy showed a strong green fluorescence signal (FIG. 18B). FT-IR analysis showed that the p24 antibody-functionalized red beads displayed many bands similar to p24 anti-IgG: the absorption bands of amide I groups at 1635 cm⁻¹ (C-O stretching vibration of peptide linkages), and the amide II groups around 1405 cm⁻¹, providing proof of the successful surface modification of the beads (FIG. 18). In addition, peaks between 2800 cm-1 and 3000 cm⁻¹ are attributable to the stretching vibrations of the alkyl groups characteristic to lipid structures that reflect the efficient surface modification of the beads with DSPE (FIG. 19D). The SDS-PAGE pattern of anti-p24 IgG showed the presence of major protein bands at 120 kDa for undigested IgG and around 50 kDa and 25 kDa, which are characteristic of IgG heavy and light chains of the digested IgG, respectively (FIG. 19E). The results for streptavidin-beads had only one protein band representing streptavidin subunits at around 13 kDa.

To prepare Pt-nanoprobes, citrate PtNPs were conjugated to monoclonal anti-biotin antibody for labeling biotinylated HIV-1 nucleic acid captured on blue beads or HIV p24 (complexed with biotinylated anti-p24 antibody) captured on red beads. We use the well-known thiol-metal bond to prepare both sets of Pt-nanoprobes (FIG. 19). Transmission electron microscopy (TEM) and the corresponding size distribution histogram show that the synthesized PtNPs are spherical with an average diameter of 4.57±1.8 nm (FIG. 19). The color of the PtNPs solution did not change after the coupling reaction and no turbidity was visible, indicating the stability of the PtNPs throughout the preparation reactions. This was confirmed using ultraviolet-visible (UV-vis) spectroscopy that showed no significant change in the absorbance spectrum of PtNPs after surface modification (FIG. 19). The efficiency of the coupling reaction of streptavidin and antibody to the surface of PtNPs was assessed by agarose gel electrophoresis and UV-vis spectroscopy. Agarose gel electrophoresis showed that the migration of PtNPs is slightly retarded post-conjugation to streptavidin or monoclonal antibody compared with unmodified PtNPs (FIG. 19), suggesting the addition of antibodies to their surface, partially interferes with electrophoretic behavior due to the different size. mass, and charge density value between PtNPs, and the antibody-PtNPs conjugates (i.e., Pt-nanoprobes). These results provide proof for the successful conjugation of streptavidin and antibody molecules on surface of beads and PtNPs.

### CamoChip Performance Testing and Validation

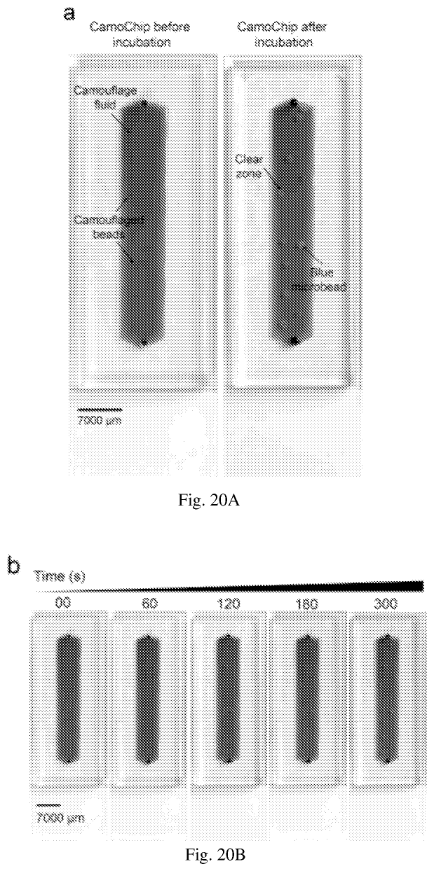

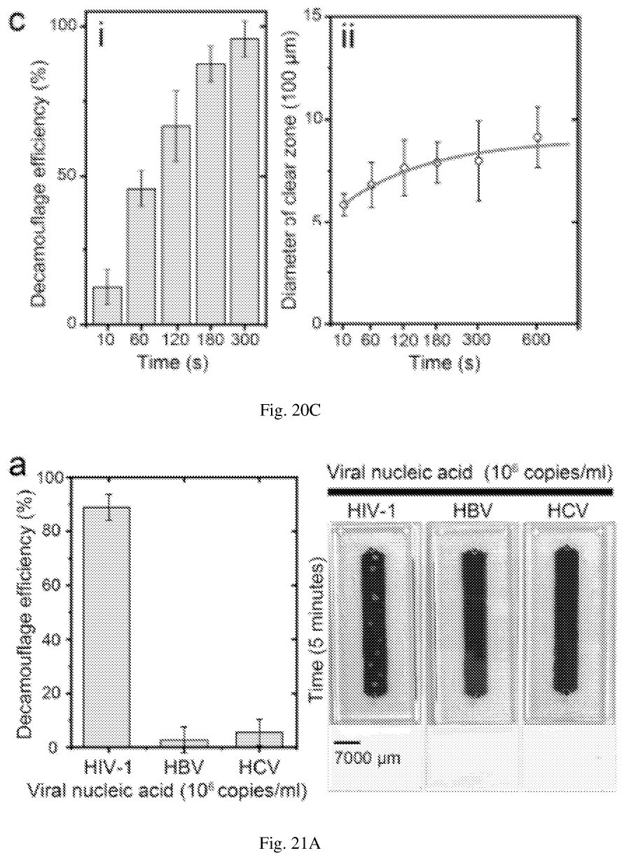

To test the proposed CamoChip, we used a microchip prepared with SA-functionalized blue beads and HIV-1 RNA as a model target (FIG. 20). RNA-spiked phosphate buffer saline (PBS) samples with a concentration of 10⁷ copies/ml were used to initially test and optimize CamoChip protocol. Samples with HIV-1 RNA were first mixed with a set of biotin-modified DNA primers that target different sequences in the HIV-1 RNA and were allowed to thermally hybridize by heating to 80° C. for 5 minutes, forming a highly biotinylated RNA target. An aliquot of the formed mixture was loaded on the chip and incubated for 15 minutes to allow nucleic acid capture on the SA-functionalized beads, followed by a washing step to remove the unbound nucleic acid. Then Pt-nanoprobes (SA-PtNPs) were loaded on chip to label the captured nucleic acid on beads. After washing, a camouflaging blue color peroxide solution is loaded on chip to mask the blue beads. Clear areas start forming and beads become visible within 5 minutes of incubation at room temperature. We noticed that the formation of clear area around the beads starts within 30 seconds of incubation at room temperature (FIG. 20). However, we optimized the incubation time to 5 minutes, the time point at which all beads and the surrounding color-free areas were fully visible and can be easily detected. We also optimized the structure of the camouflaging peroxide solution to have 10% H₂O₂. We tested different concentrations of H₂O₂ and 10% was the concentration that provided the optimum dynamics for gas formation and accumulation around the bead in the form of a clear area. We also tested different bead surface coverage ratios with PEG-DSPE ligand to create a partial hydrophobicity, which is needed to facilitate repelling liquid and allowing gas to collect around beads. We noticed that 20% coverage of bead's surface is necessary for the generated O₂ gas to form color-clear area.

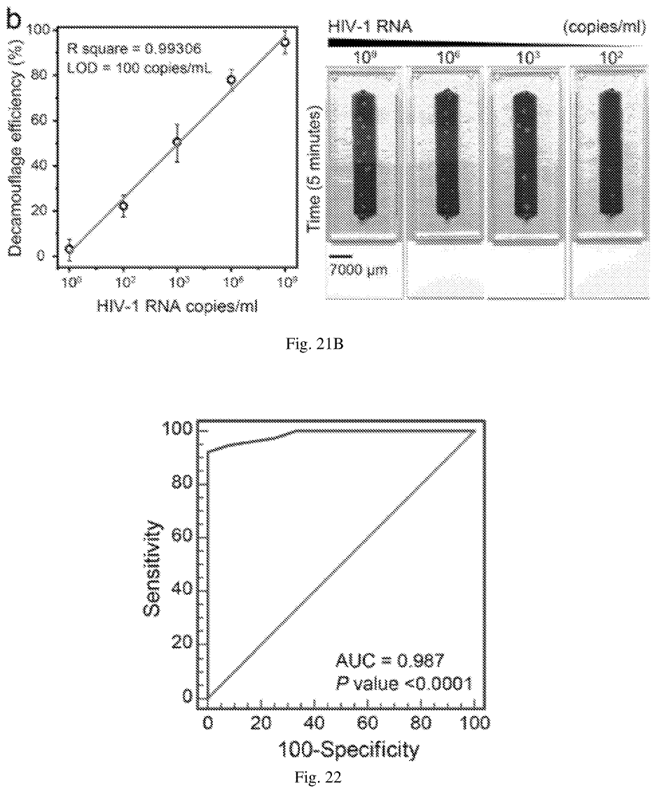

To test the detection specificity of the optimized CamoChip, we tested the performance of chip loaded with SA-functionalized blue beads in the presence of the target HIV-1 RNA and other non-target viruses, including hepatitis C virus (HCV) RNA and hepatitis B virus (HBV) DNA at concentration of 10⁶ copies/ml (FIG. 21). All the beads remain unmasked with the colored peroxide solution after incubation >5 minutes with all the non-target viruses, confirming the specificity of the developed CamoChip for target HIV-1 RNA testing.

To test the detection sensitivity of the optimized CamoChip, we tested the performance of chip loaded with SA-functionalized blue beads with serial dilutions of HIV-1 RNA from 10⁹ to 10¹ copies/ml. Each dilution was mixed with a set of biotin-modified oligonucleotides and tested using the optimized CamoChip protocol. The number of beads and the size of the formed clear zone around the beads was measured and correlated to the tested HIV-1 RNA concentration. The results indicated that the number of beads that become visible increases with increasing the tested RNA concentration. More than 90% of beads become visible at the highest tested concentration of 10⁹ copies/ml and the limit of detection was around 100 copies/ml. In addition, the average percentage of decamouflaging efficiency proportionally increased with the tested concentration, with a minimum value of 22.2±4.8% of beads at 100 copies/ml (FIG. 21C).

To validate the performance of CamoChip, we used 50 samples of PBS and human plasma spiked with HIV-1 RNA at final concentrations that range from 10⁰ to 10⁹ copies/ml (FIG. 21). Samples were tested using the optimized CamoChip in parallel with the ‘gold standard’ techniques of RT-PCR. Receiver operating characteristic (ROC) analysis showed that HIV-1 RNA concentration >80 copies/ml yields an optimum sensitivity of 92.11% with a CI of 78.6-98.3% and a specificity of 100% with a CI of 73.5-100% for CamoChip. At this threshold, the area under the curve (AUC) was 0.987, with an exact binomial CI ranging from 0.905 to 1.0 (FIG. 22).

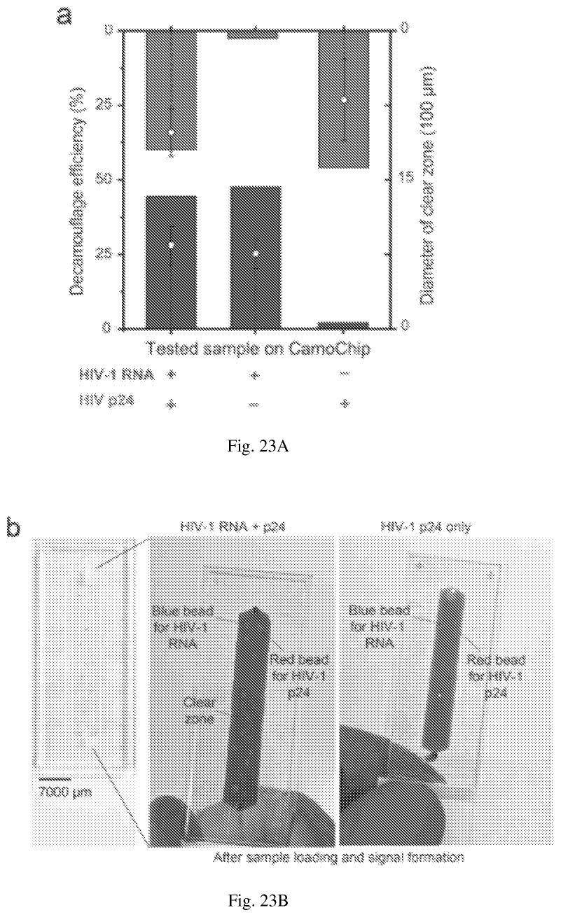

To determine the efficiency of the developed CamoChip for multiplex testing, we used HIV-1 RNA and HIV-1 p24 antigen as model targets. CamoChip was prepared with SA-functionalized blue beads and anti-HIV p24 polyclonal antibody-functionalized red beads loaded on the chip and incubated for 15 minutes. After washing, a mixture of biotinylated HIV-1 RNA (at a concentration of 10⁶ copies/ml) and HIV-1 p24 complexed with biotinylated anti-p24 mAb (at a concentration of 1 ng/ml) was loaded on chip for target capture. Then the captured targets were labeled with Pt-nanoprobes (i.e., PtNPs modified with anti-biotin mAb). Then the microchip was loaded with the colored peroxide solution and incubated for 5 minutes. We were able to visually detect the blue and red beads and the formed clear area (FIG. 23).

### Example 2

### Multi-Arm Primed Looping and Amplification of Nucleic Acid

In this example, we describe the development of simple and easy-to-design, rapid, and specific isothermal amplification methods of nucleic acids that can significantly reduce the risk and challenges associated with their implementation into advanced platforms for nucleic acid analysis and testing. The novel isothermal amplification method that uses a relatively simplified design with a multi-arm polymer-based DNA primer to rapidly amplify the target nucleic acid with high efficiency and specificity. The novel multi-arm primer-based amplification (MAPA) method for nucleic acid rapidly amplifies the target nucleic acid with high efficiency under isothermal conditions. This method relies on using a unique set of three DNA primers that target a total of four distinct sequences in the target DNA. One multi-arm DNA primer designed with eight arms of polyethylene glycol (PEG), each arm carries a copy of a single-stranded (ss) DNA that either complements the sense or anti-sense sequence of the target DNA (n=6 with DNA of 50:50% sense to anti-sense and 2 with biotin group). Two single-stranded DNA primers complement sequences at the start and end of the target DNA. The MAPA reaction starts with the anti-sense arms binding to the complementary sequence in the target DNA, followed by the formation of looped-arm dsDNA structures, mediated by the arms carrying the sense sequences. This serves as a template for DNA synthesis via continuous cycles of alternating looping and unlooping steps and the formation of muti-arm amplicons that are consumed in the elongation step of the reaction, forming large muti-amplicon DNA structures. These amplicons are developed in <30 minutes, allowing for rapid and specific detection of nucleic acid targets such as human immunodeficiency virus-1 (HIV-1) with a sensitivity down to 10³ copies/ml.

### Material and Methods

### Target Sequence and DNA Oligonucleotides

The nucleic acid target of the HIV-1 pol-integrase gene, a well-conserved region of the HIV-1 genome, was used as a template for MAPA development and testing. The HIV-1 target sequence was prepared and cloned in pUCIDT-AMP using the Gene Synthesis service from Integrated DNA Technologies, Inc. A set of three primers were designed based on the sequence of the target region available under the GenBank accession number K02013 (4720-4910). Initiator primer (IP) with 15-mer targets the sequence from 4720 to 4720: 5′-GGTAAGAGATCAGGCTGAACATC-3′ (SEQ ID NO: 1). The multi-arm DNA looping primer (LP) consisted of the complementary sequence of L1 (20-mer from 4720 to 4720; 5-/5ThioMC6-D/AGACAGCAGTACAAATGGCA-3) (SEQ ID NO: 2) and L2 (CCCCAATCCCCCCTTTTCTT/3ThioMC3-D) (SEQ ID NO: 3) that are thiolated on their 3′ and 5′, respectively. Two DNA unlooping primers (ULP and ULP′) 15 mer in size were tested in this study. ULP targets the sequence from 4720 to 4720: 5′-CTGCTGTCCCTGTAATAAACCC-3′, (SEQ ID NO: 4) while ULP′ targets and the sequence from 4720 to 4720: 5′-AGTGCAGGGGAAAGAATAGTAGAC-3′ (SEQ ID NO: 5).

### Multi-Arm DNA Primer Synthesis and Characterization

Multi-arm looping DNA primers were prepared of 8-arm heterobifunctional PEG molecules that have two arms ending with biotin groups and six arms with maleimide groups readily accessible coupling with thiolated DNA oligonucleotides (i.e., L1 and L2) through the well-known thiol-maleimide conjugation chemistry. The detailed protocol includes three main steps: (1) DNA activation, (2) DNA coupling to the multi-arm PEG molecules, and (3) DNA primer washing and concentration. The activation of thiolated L1 and L2 was performed using a tris(2-carboxyethyl) phosphine (TCEP)-reduction step for 2 hours at room temperature. The reduced DNA oligonucleotides (i.e., L1 and L2 with free thiol groups) are mixed with PEG solution at a ratio of 50:1 and incubated for 2 hours in phosphate buffer pH 7.4. The prepared multi-arm DNA-PEG conjugates are washed and concentrated using 10 KDa centrifugal filter units. The prepared multi-arm PEG-DNA primer conjugates were characterized using agarose gel electrophoresis, UV-vis, and FT-IR spectroscopy techniques.

### MAPA Reaction and Condition

The MAPA reaction was carried out in a 25 μl volume (total), containing the following components: 0.4 μM of IP primer, 1.6 μM of each LP and ULP primers, 0.4M betaine (Sigma-Aldrich, St. Louis, MO), 10 mM MgSO4, 1.4 mM dNTPs, 1× ThermoPol reaction buffer (New England Biolabs, Ipswich, MA), 8U Bst DNA polymerase (New England Biolabs), and 1 μl of the target nucleic acid. The MAPA amplification was performed using a Bio-Rad T100 Thermal Cycler (Bio-Rad, Foster City, CA). The reaction mixture was heated at 65° C. for 30 minutes and then held at 90° C. for 3 minutes to terminate the reaction. Negative controls were included in each run, including a technical control to check for cross-contamination. Identification of amplified DNA product was determined by gel electrophoresis on a 2% agarose gel, followed by staining with GelRed stain and visualization on a UV transilluminator.

### Amplification Specificity and Sensitivity of HIV-1

We used a sequence of HIV-1 RNA cloned in pUCIDT-AMP to test the specificity and sensitivity of MAPA reaction. We optimized the MAPA reaction by evaluating a range of amplification temperatures (65° C.-69° C.). Additionally, the minimum amplification time required for maximum sensitivity was tested at different time points. The target DNA was added to the reaction in 10-fold dilutions, and amplification was performed for 20, 25, 30, 35 or 40-minutes. Amplification specificity was tested against FABP4 and FABP3 plasmids as non-specific targets and using the optimized MAPA reaction condition.

### Results and Discussion

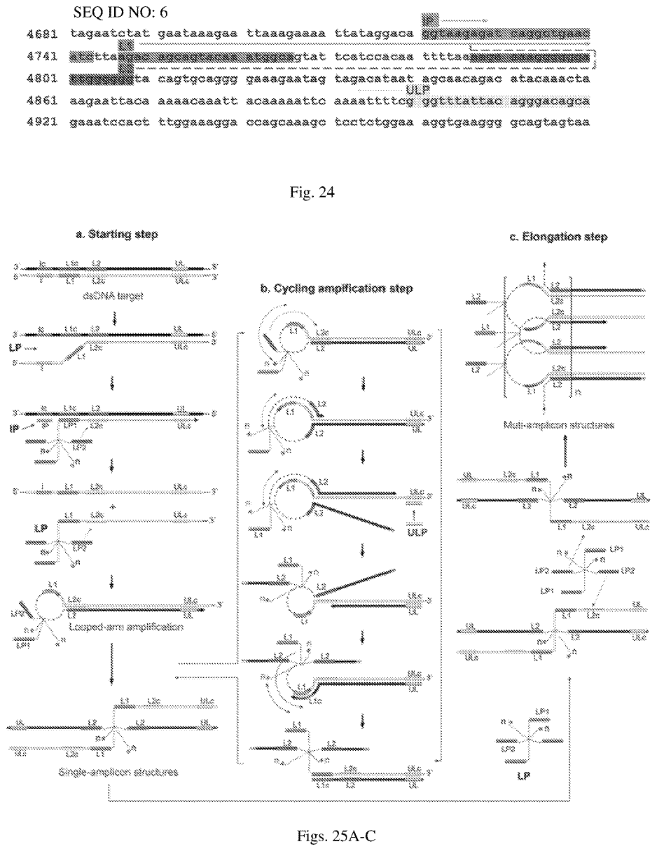

We developed an isothermal nucleic acid amplification reaction that relies on using uniquely designed multi-arm DNA primers to amplify the target nucleic acid sequence with high specificity and sensitivity. Along with a pair of primers (i.e., IP and ULP), these arms are expanded into a multi-arm amplicon that eventually elongated into a muti-amplicon structure. The DNA arms of the developed primers are designed with anti-sense and sense DNA oligonucleotides (each is 20-mer in length) that specifically complement two distinct but subsequent sequences in the target DNA (FIG. 24). Multiple copies (n=3) of each DNA oligonucleotides are linked to a highly-branched (n=8, 3 arms with the sense DNA sequence, three arms with the anti-sense DNA sequence, and two arms are terminally modified with biotin group) polymer molecule of polyethylene glycol (PEG) that connects the different DNA arms and provide support for dynamic and flexible interaction with the target DNA (FIG. 25). The DNA sequences are designed in a way that the anti-sense DNA arms prime for the amplification, followed by self-priming with the sense DNA arms (linked to the anti-sense arms by PEG), generating a stem-looped PEG-DNA hybrid structure, in which the loops are partially made of ssDNA and two arms PEG. The other free PEG arms with DNA (n=4) mediate a chain of self-looping, leading to the formation of multi-arm amplicons, which are eventually used as templates in the elongation step to generate large muti-amplicon structures. In the self-looping step, the free DNA arm with the anti-sense sequence turns over the existing loop (benefiting from the flexibility and extendibility of PEG as backbone polymer) to self-prime for the formation of a new strand and stem-looped structure. The self-looping cycle continues till all the DNA arms with the sense sequence (L2) expand into a copy of the target sequence. Simultaneously, the binding of the unlooping primer (ULP) stimulates unlooping of the expanded arms and the formation of a sense DNA strand—with a complementary sequence of the anti-sense arm (Lc1), which provides an amplification template for expanding the sense arms (L1), and the subsequent formation of multi-arm amplicon structures (FIG. 25).

To synthesize the multi-arm DNA primers, we relied on the well-known maleimide-thiol coupling chemistry to guide a controlled, directional conjugation of the L1 and L2 DNA oligonucleotides with PEG. We controlled the molar ratio of coupling reaction to 1 PEG:50 DNA oligonucleotide (of each L1 and L2) to confirm and excess availability of DNA to bind with the PEG and at an equal ratio of L1 to L2. In our primer design, we used 8-arm PEG (with six maleimide and two biotin termini) with a relatively high molecular weight of 20 KDa to provide enough arm-length (100-200 nm) necessary for accessible and dynamic binding and to unbind during the amplification process of the target DNA using Bst polymerase. The efficiency of the PEG-DNA coupling reaction was assessed using magnetic separation and agarose gel electrophoresis. The results indicated that the streptavidin (SA)-modified magnetic beads were able to bind to the free biotin groups on the PEG-DNA primers (L1 and L2), and the formed MB-PEG-DNA conjugates were large to migrate in agarose gel and appeared concentrated in the sample loading wells in the agarose gel, with a smeared fluorescence signal for PEG-DNA. On the contrary, SA-modified magnetic beads reacted with only DNA oligonucleotides (L1 and L2, without PEG) and were able to migrate in the gel, and the DNA primers appeared in a fully separated band on the gel (FIG. 26). In addition, the results of UV-vis spectroscopy analysis of the washed multi-arm DNA primers (i.e., PEG-DNA conjugates) confirmed the presence of PEG characteristic absorbance peak around 112 nm and DNA oligonucleotide absorbance peak around 272 nm, confirming the stable coupling of PEG and DNA and formation of the muti-arm DNA primers (FIG. 26B).

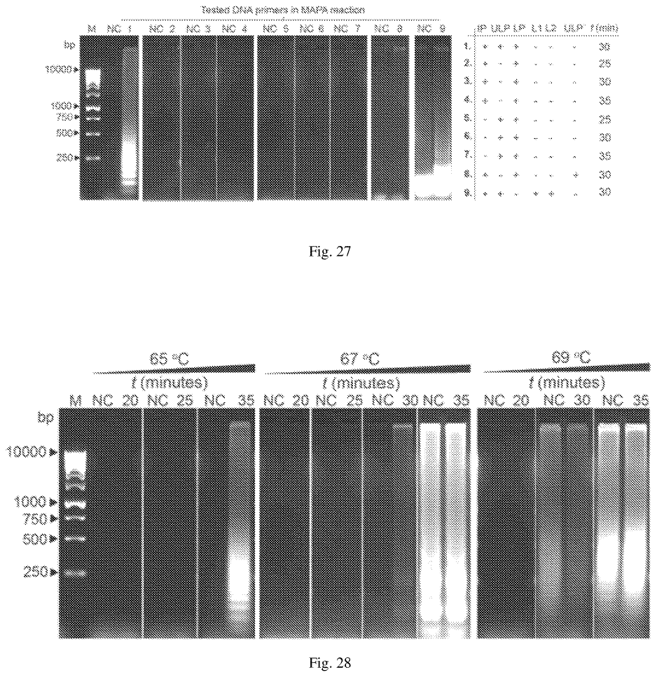

To test the activity of the developed multi-arm DNA primers, we prepared a set of MAPA reaction mixtures that included (i) DNA polymerase, (ii) a pair of DNA primers IP and ULP, (iii) deoxyribonucleotide triphosphate (dNTPs) and (iv) a magnesium salt of MgSO4 mixed in 1× isothermal amplification buffer. We used Bst polymerases to provide high processivity at a relatively high reaction temperature (60° C.-70° C.) to avoid the non-specific binding of primers that might occur with polymerases that require a lower optimum temperature. The IP and ULP primers are chosen with melting temperature (Tm) values in the same range of both the sense and anti-sense DNA oligonucleotides used in the preparation of the muti-arm looping primers (54° C.-57° C.). However, it is worth mentioning that the Tm value of the L2 sequence (looping arms) used in the muti-arm primer was set slightly higher than those of IP and ULP in order that a looped-out structure formed immediately after the release of the single-stranded DNA from the template. Furthermore, both LP and ULP were used at the same concentration and at a significantly higher concentration of IP (20%<LP or ULP) to ensure that the synthesis of stem-looped PEG-DNA structures occurred earlier from the initiator primers. While LP and ULP are equally added in the reaction to promote simultaneous looping and unlooping steps in the cycling steps—required for effective amplification of both the sense and anti-sense DNA arms and the subsequent formation of the muti-arm amplicons. Following this design, we tested the efficiency of the MAPA reaction to amplify the HIV-1 sequence as a model DNA template. The results indicated that was only amplification in the presence of the target, while no amplification was detected in the control samples. The formed amplicons in the presence of the target appeared with a unique smear-like pattern, indicating the formation of a large amplicon along with multiple DNA bands with variable lengths (<250 bp). This initial testing step of MAPA was performed at 65° C., which is the average optimum temperature for the Bst enzyme and slightly higher than primers Tm to ensure specific and rapid target amplification. Afterward, we tested the effect of each primer, and the amplification products were visualized using agarose gel electrophoresis (FIG. 27). The results indicated that there is no amplification was detected in the absence of IP or ULP, even with extended amplification reaction times up to 35 minutes, confirming the curial rule of both primers in the amplification process. In addition, we tested the efficiency of MAPA to amplify the target DNA using free L1 and L2 DNA oligonucleotides and without multi-arm PEG (no arms), and the results showed a strong non-specific amplification (FIG. 27). This confirms the significant relationship between the presence of muti-arm PEG for effective looping and specific target amplification. Although this looping is crucial for the cycling step in MAPA reaction, the amplification process can proceed through an alternative pathway that relies on the formation of multi-looping instead of self-looping. Although this thermodynamically can be more challenging than the self-looping, we tested this possibility by using ULP′ that complements the antisense strand in the formed amplicon and is required for the muti-looping steps. The results are presented in FIG. 27C, indicating specific but significantly reduced amplification when compared to MAPA using ULP that promotes the self-looping and unlooping-based amplification.

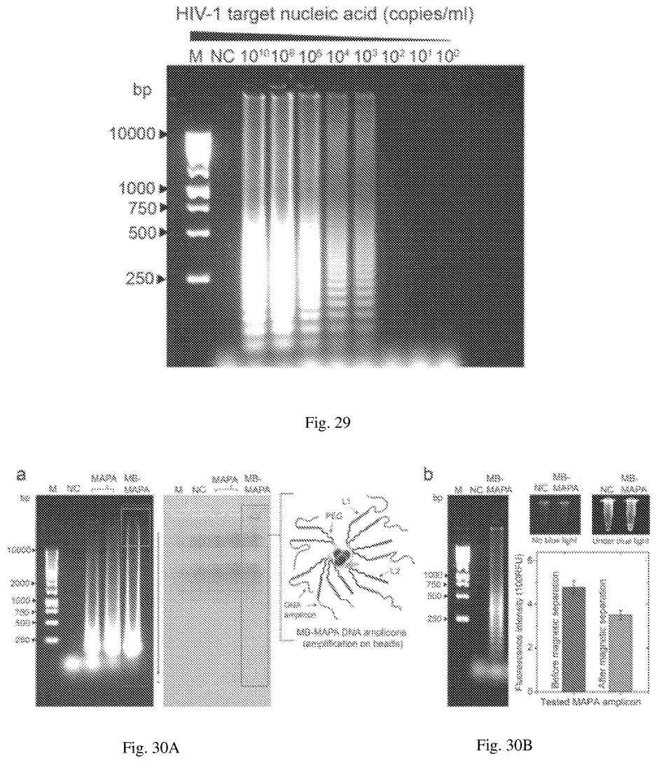

To further optimize the MAPA reaction conditions for sensitive HIV-1 detection, we tested the effect of different reaction temperatures (within the temperature range of the Bst enzyme) and at time points ranging from 10-60 minutes. The results indicated a significant increase in the amplification rate with increasing the reaction temperature up to 67° C., and specific target amplification was detectable in 10 minutes after amplification. However, the use of a relatively high reaction temperature of around 70° C. increased the chance for non-specific amplification, and DNA was detectable in control samples (no HIV-1 target) at 30 minutes (FIG. 28). Subsequently, we used MAPA reaction to test different concentrations of the target HIV-1 sequence cloned in pUCIDT-AMP at the optimized reaction temperature of 67° C. We were able to detect concentrations down to 103 copies/ml, which confirms a relatively sensitive target detection without additional optimization steps (FIG. 29).

We developed a rapid isothermal nucleic acid amplification reaction that can effectively amplify the target sequence within 30 minutes. The developed MAPA reaction deploys the PEG multi-arm polymeric structure to promote a rapid looping and priming of the DNA synthesis of amplicons. PEG is a widely used biocompatible hydrophilic polymer with highly flexible chains that provide the conformational accessibility for DNA primers to bind to the target sequence. We attempted to replace the PEG function to link the L1 and L2 DNA primers with streptavidin molecules and form multi-arm primers (FIG. 29). SA molecules as homo-tetramers with exceptional binding affinity to biotin groups have the potential to link biotinylated L1 and L2 sequences. MAPA reaction performed using the SA-based multi-arm primers showed a significantly reduced amplification when compared with PEG-based primers, confirming the importance of PEG chain length and flexibility to render effective template-primer binding and DNA amplification. Being connected to muti-arm structures favor, at least thermodynamically, the priming and the rate of the DNA synthesis allowing for the rapid formation of large amplicons in a relatively short time. Although we used a relatively challenging target such as the HIV-1 genome, we were able to detect amplification products on agarose gel within 10 minutes at 106 copies/ml. Compared to other isothermal methods that adopt DNA looping for target amplification, including LAMP, our MAPA reaction requires a significantly smaller number of priming sequences (4 in MAPA and 6-8 in LAMP), shorter amplification time (S 30 minutes), and comes with a similar sensitivity limit. In addition, the use of PEG-based DNA primers provides a unique opportunity to engineer the produced amplicons to enable highly sensitive and reliable applications without additional or independent steps. In the current design of MAPA reaction, we used biotinylated PEG molecules that provide the chance for amplicon separation without additional biotinylation or amplicon processing steps (after reaction). We were able to amplify the target HIV-1 sequence in the presence of SA-modified magnetic nanobeads (added at the start of the reaction). The successful amplification and capturing of the formed amplicons on the surface of beads were confirmed using agarose gel electrophoresis. The formed DNA amplicon-MB complexes were large enough in size to freely move in the agarose gel (5%), and a major part of the amplification product was shifted with the MB trapped in the sample loading wells on the top of the gel (FIG. 30). Also, the spontaneous biotinylation of amplicons allowed us to magnetically separate the target-generated amplicons from any non-specific background amplification for highly specific and reliable target detection (FIG. 30B). This is one of the major drawbacks of the existing isothermal amplification methods. These results together support the use of PEG-based and PEG-like polymers to bring more functions and engineering to nucleic acid amplification steps that eventually could promote its integrability and implementation into advanced devices and systems for nucleic acid analysis and testing.

Isothermal nucleic acid amplification holds great potential for advancing molecular testing and clinical analysis. Any significant reduction in the amplification reaction time and the need for time and steps (i.e., more simple protocol) will shorten the path to their technical maturity and commercialization. Our reported technology allows rapid target amplification with a significantly simplified amplification protocol that uses a minimum number of primers and polymerase enzymes without compromising the reaction sensitivity and specificity. This is a major advancement in the area of nucleic acid research that can impact the development of point-of-care diagnostics, genome sequencing, single cell testing, rapid pathogen testing, and clinical and environmental analyses.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: An apparatus for detection of an analyte, the apparatus comprising:
a first detectable agent having one or more detectable characteristic; and
a masking agent having one or more characteristic that masks the detection of the one or more characteristic of the first detectable agent in absence of the analyte; wherein at least one of the first detectable agent or the masking agent is configurable in the presence of the analyte to allow detection of the one or more characteristic of the first detectable agent which is indicative of the presence of the analyte, wherein the first detectable agent is configured to specifically bind to the analyte and the one or more characteristic of the first detectable agent is undetectable in the presence of the masking agent when the first detectable agent is not bound to the analyte and detectable upon binding to the analyte.

2. (canceled)

3. (canceled)

4: The apparatus of claim 1, wherein binding of the analyte to the first detectable agent triggers configuration of the masking agent to form an unmasked or mask-free signal zone that allows detection of the first detectable agent.

5: The apparatus of claim 4, wherein the masking agent comprises a masking fluid that masks the one or more characteristic of the first detectable agent and binding of the analyte to the first detectable agent disperses masking fluid around the first detectable agent to allow detection of the first detectable agent which is indicative of the presence of the analyte.

6. (canceled)

7. (canceled)

8: The apparatus of claim 5, wherein the first detectable agent includes one or more first detectable particles affixed to a surface of a substrate, the first detectable particles being configured to specifically bind the analyte and the one or more first detectable particles affixed to the surface of the substrate are covered with the masking fluid in an absence of the analyte.

9. (canceled)

10: The apparatus of claim 8, further comprising a detection agent that specifically binds to the analyte to form a detection agent-analyte-particle complex, the detection agent being masked by the masking fluid when the analyte is not bound to the detectable particle and the detection agent.

11: The apparatus of claim 10, wherein the masking fluid visually masks the first detectable particles that do not form the detection agent-analyte-particle complex.

12: The apparatus of claim 11, wherein the detection agent of the complex catalyzes conversion of the masking fluid into a gas that disperses masking fluid around the complex to form the fluid-free zone and allowing optical detection of the complex on the substrate.

13: The apparatus of, claim 11 wherein the first detectable particles comprise a plurality of microbeads and the microbeads have a diameter of about 10 μm to about 1 mm.

14. (canceled)

15: The apparatus of claim 10, wherein the first detectable particles include a capturing agent on outer surfaces of the first detectable particles that specifically bind to the analyte.

16: The apparatus of claim 15, wherein the capturing agent includes at least one of a small molecule, nucleotide, protein, antibody or streptavidin.

17. (canceled)

18: The apparatus of claim 16, wherein the capturing agent includes streptavidin and the analyte includes a biotin-modified nucleic acid or peptide.

19. (canceled)

20: The apparatus of claim 10, wherein the detection agent includes platinum nanoparticles (PtNPs).

21: The apparatus of claim 20, wherein the PtNPs are conjugated to streptavidin, or anti-biotin antibodies.

22: The apparatus of claim 8, wherein the masking fluid has a color that visually masks the first detectable particles appearance upon loading of the masking fluid onto the surface of the substrate.

23: The apparatus of any of claim 8 to 22, wherein the masking fluid includes a peroxide solution.

24. (canceled)

25: The apparatus of claim 22, wherein the substrate includes at least one microchannel that extends along a portion of the substrate, the microchannel including the first detectable particles and masking fluid.

26. (canceled)

27: The apparatus of claim 8, further comprising one or more second detectable agent, the second detectable agent including second detectable particles affixed to the surface of the substrate and the second detectable particles include a second plurality of second capture agents to specifically bind a second analyte.

28. (canceled)

29: The apparatus of claim 27, further comprising a second detection agent that specifically bind the second analyte to form a second detection agent-analyte-particle complex that forms a fluid-free zone around the second agent-analyte-particle complex allowing for detection of the second agent-analyte-particle complex on the substrate.

30: The apparatus of claim 29, wherein the one or more second detectable particles are a substantially different color than the first detectable particles.

31-33. (canceled)

34: A method of detecting an analyte, the method comprising:
providing an apparatus of claim 8;
loading a sample comprising the analyte onto the surface of the substrate to bind the analyte to the outer surface of one or more first detectable particles;
labeling the bound analyte with a plurality of first detection agents to form a first detection agent-analyte-particle complex on the surface of the substrate;
loading a masking fluid onto the surface of the substrate to mask the one or more first detectable particles, wherein the first detection agent of the complex catalyzes degradation of the masking fluid to form a masking fluid-free zone around the particle of the complex; and
detecting the one or more first detectable particles in the masking fluid-free zone, wherein detection of the one or more first detectable particles in the masking fluid-free zone is indicative of the presence of the analyte in the sample.

35-53. (canceled)
